(19) United States
(12) Patent Application Publication
(10) Pub. No.: US 2006/0240486 A1
(43) Pub. Date: Oct. 26, 2006

(54) Immunoprecipitation-based assay for predicting in vivo efficacy of beta-amyloid antibodies

(76) Inventors: Kelly Lee Johnson-Wood, Scotts Valley, CA (US); Peter Andrew Seubert, San Francisco, CA (US); Jack Steven Jacobsen, Ramsey, NJ (US)

Correspondence Address: LAHIVE & COCKFIELD, 28 STATE STREET, BOSTON, MA, 02109, US

(21) Appl. No.: 11/304,072

(22) Filed: Dec. 15, 2005

Related U.S. Application Data
(60) Provisional application No. 60/636,687, filed on Dec. 15, 2004.
(60) Provisional application No. 60/736,045, filed on Nov. 10, 2005.

(51) Int. Cl. G01N 33/567; G01N 33/53

(52) U.S. Cl. 435/7.2

(57) ABSTRACT

In various aspects, the present invention provides methods and kits for predicting the therapeutic efficacy of an immunological reagent, identifying an immunological reagent having therapeutic efficacy, or both, for the treatment of an amyloidogenic disorder by comparing the amount of Aβ monomer in an Aβ preparation which binds to the immunological reagent to an amount of one or more Aβ oligomers in the Aβ preparation which bind to the immunological reagent to determine a relative bound amount, and predicting the efficacy of the immunological reagent, identifying an immunological reagent having therapeutic efficacy, or both, for the treatment of an amyloidogenic disorder based at least on the relative bound amount.

### RELATED APPLICATIONS

This application claims the benefit of priority to prior-filed provisional patent applications U.S. Ser. No. 60/636,687, filed Dec. 15, 2004, and U.S. Ser. No. 60/736,045, filed Nov. 10, 2005, both entitled “AN IMMUNOPRECIPITATION-BASED ASSAY FOR PREDICTING IN VIVO EFFICACY OF BETA-AMYLOID,” the entire contents of which are incorporated herein by reference.

### BACKGROUND OF THE INVENTION

Alzheimer's disease (AD) is a progressive disease resulting in senile dementia. See generally Selkoe, TINS 16:403 (1993); Hardy et al., WO 92/13069; Selkoe, J. Neuropathol. Exp. Neurol. 53:438 (1994); Duff et al., Nature 373:476 (1995); Games et al., Nature 373:523 (1995). Broadly speaking, the disease falls into two categories: late onset, which occurs in old age (65+ years) and early onset, which develops well before the senile period, i.e., between 35 and 60 years. In both types of disease, the pathology is the same but the abnormalities tend to be more severe and widespread in cases beginning at an earlier age. The disease is characterized by at least two types of lesions in the brain, neurofibrillary tangles and senile plaques. Neurofibrillary tangles are intracellular deposits of microtubule associated tau protein consisting of two filaments twisted about each other in pairs. Senile plaques (i.e., amyloid plaques) are areas of disorganized neuropil up to 150 μm across with extracellular amyloid deposits at the center which are visible by microscopic analysis of sections of brain tissue. The accumulation of amyloid plaques within the brain is also associated with Down's syndrome and other cognitive disorders.

The principal constituent of the plaques is a peptide termed Aβ or β-amyloid peptide. Aβ peptide is an approximately 4-kDa internal fragment of 39-43 amino acids of a larger transmembrane glycoprotein named protein termed amyloid precursor protein (APP). As a result of proteolytic processing of APP by different secretase enzymes, Aβ is primarily found in both a short form, 40 amino acids in length, and a long form, ranging from 42-43 amino acids in length. Part of the hydrophobic transmembrane domain of APP is found at the carboxy end of Aβ, and may account for the ability of Aβ to aggregate into plaques, particularly in the case of the long form. Accumulation of amyloid plaques in the brain eventually leads to neuronal cell death. The physical symptoms associated with this type of neural deterioration characterize AD.

Several mutations within the APP protein have been correlated with the presence of AD. See, e.g., Goate et al., Nature 349:704 (1991) (valine⁷¹⁷ to isoleucine); Chartier Harlan et al., Nature 353:844 (1991) (valine⁷¹⁷ to glycine); Murrell et al., Science 254:97 (1991) (valine⁷¹⁷ to phenylalanine); Mullan et al., Nature Genet. 1:345 (1992) (a double mutation changing lysine⁵⁹⁵-methionine⁵⁹⁶ to asparagine⁵⁹⁵-leucine⁵⁹⁶). Such mutations are thought to cause AD by increased or altered processing of APP to Aβ, particularly processing of APP to increased amounts of the long form of Aβ (i.e., Aβ1-42 and Aβ1-43). Mutations in other genes, such as the presenilin genes, PS 1 and PS2, are thought indirectly to affect processing of APP to generate increased amounts of long form Aβ (see Hardy, TINS 20: 154(1997)).

Mouse models have been used successfully to determine the significance of amyloid plaques in AD (Games et al., supra, Johnson-Wood et al., Proc. Natl. Acad. Sci. USA 94:1550 (1997)). In particular, when PDAPP transgenic mice, (which express a mutant forma of human APP and develop AD pathology at a young age), are injected with the long form of Aβ, they display both a decrease in the progression of AD pathology and an increase in antibody titers to the AD peptide (Schenk et al., Nature 400, 173 (1999)). The above findings implicate Aβ, particularly in its long form, as a causative element in AD.

Aβ peptide can exist in solution and can be detected in the central nervous system (CNS) (e.g., in cerebral spinal fluid (CSF)) and plasma. Under certain conditions, soluble Aβ is transformed into fibrillary, toxic, β-sheet forms found in neuritic plaques and cerebral blood vessels of patients with AD. Several treatments have been developed which attempt to prevent the formation of Aβ peptide, for example, the use of chemical inhibitors to prevent the cleavage of APP. Immunotherapeutic treatments have also been investigated as a means to reduce the density and size of existing plaques. These strategies include passive immunization with various anti-Aβ antibodies that induce clearance of amyloid deposits, as well as active immunization with soluble forms of Aβ peptide to promote a humoral response that includes generation of anti-Aβ antibodies and cellular clearance of the deposits. Both active and passive immunization have been tested as in mouse models of AD. In PDAPP mice, immunization with Aβ was shown to prevent the development of plaque formation, neuritic dystrophy and astrogliosis. Treatment of older animals also markedly reduced the extent and progression of these AD-like neuopathologies. Shenk et al., supra. Aβ immunization was also shown to reduce plaques and behavioral impairment in the TgCRND8 murine model of AD. Janus et al. (2000) Nature 408:979-982. Aβ immunization also improved cognitive performance and reduced amyloid burden in Tg 2576 APP/PSI mutant mice. Morgan et al. (2000) Nature 408:982-985. Passive immunization of PDAPP transgenic mice has also been investigated. It was found, for example, that peripherally administered antibodies enter the central nervous system (CNS) and induced plaque clearance in vivo. Bard et al. (2000) Nat. Med. 6:916-919. The antibodies were further shown to induce Fc receptor-mediated phagocytosis in and ex vivo assay. Antibodies specific for the N-terminus of Aβ42 have been demonstrated to be particularly effective in reducing plaque both ex vivo and in vivo. See U.S. Pat. No. 6,761,888 and Bard et al. (2003) Proc. Natl. Acad. Sci. USA 100:2023-2028. Antibodies specific for the mid-region of Aβ42 also showed efficacy. U.S. Pat. No. 6,761,888

Two mechanisms are proposed for effective plaque clearance by immunotherapeutics, i.e., central degradation and peripheral degradation. The central degradation mechanism relies on antibodies being able to cross the blood-brain barrier, bind to plaques, and induce clearance of pre-existing plaques. Clearance has been shown to be promoted through an Fc-receptor-mediated phagocytosis (Bard, et al. (2000) Nat. Med. 6:916-19). The peripheral degradation mechanism of Aβ clearance relies on a disruption of the dynamic equilibrium of Aβ between brain, CSF, and plasma by anti-Aβ antibodies, leading to transport of Aβ from one compartment to another. Centrally derived Aβ is transported into the CSF and the plasma where it is degraded. Recent studies have concluded that soluble and unbound Aβ are involved in the memory impairment associated with AD, even without reduction in amyloid deposition in the brain. Further studies are needed to determine the action and/or interplay of these pathways for Aβ clearance (Dodel, et al., The Lancet, 2003, 2:215)

While the majority of treatments to date have been aimed at reducing amyloid plaque buildup, it has been recently noted that certain cognitive impairments (e.g. hippocampal-dependent conditioning defects) associated with amyloidogenic disorders begin to appear before amyloid deposits and gross neuropathology are evident (Dineley et al., J. Biol. Chem., 2002, 227: 22768). Furthermore, while the pathogenic role of amyloid peptide aggregated into plaques has been known for many years, the severity of dementia or cognitive deficits is only somewhat correlated with the density of plaques whereas a significant correlation exists with the levels of soluble Aβ. (see, e.g., McLean et al., Ann Neurol, 46:860-866 (1999). Some studies have shown or suggested that soluble Aβ oligomers are implicated in synaptotoxicity and memory impairment in APP transgenic mice due to mechanisms including increased oxidative stress and induction of programmed cell death. (See, e.g., Lambert, et al., (1998), PNAS, 95: 6448-53; Naslund et al., (2000), JAMA, 283: 1571; Mucke et al., J. Neurosci, 20:4050-4058 (2000); Morgan et al., Nature, 408:982-985 (2000); Dodart et al., Nat Neurosci, 5:452-457 (2002); Selkoe et al., (2002), Science, 298: 789-91; Walsh et al., Nature, 416:535-539 (2002)). These results indicate that neurodegeneration may begin prior to, and is not solely the result of, amyloid deposition. Accordingly, there exists the need for new therapies and reagents for the treatment of AD, in particular, therapies and reagents capable of effecting a therapeutic benefit via intervention with various mechanisms of Aβ-induced neurotoxicity.

### SUMMARY OF THE INVENTION

In various aspects, the present invention features methods for identifying immunological reagents having therapeutic efficacy for the treatment of one or more amyloidogenic disorders (e.g., Alzheimer's disease), or combination of amyloidogenic disorders. The methods are based, at least in part, on a comparison of the binding of one or more Aβ oligomers in an Aβ preparation to an immunological reagent to the binding of Aβ monomers in the Aβ preparation to the immunological reagent. The one or more Aβ oligomers can include, for example, one or more of Aβ dimers, Aβ trimers, Aβ tetramers, and Aβ pentamers. The comparison of the binding to an immunological reagent is used, at least in part, to identify an immunological reagent as having (or not having) therapeutic efficacy for the treatment of one or more amyloidogenic disorders. In particular, the invention features identification of Aβ antibodies having therapeutic efficacy for the treatment of Alzheimer's disease (AD).

In various embodiments, an immunological reagent is identified as having therapeutic efficacy for the treatment of one or more amyloidogenic disorders when the binding of one or more Aβ oligomers in the Aβ preparation to the immunological reagent is greater than the binding of Aβ monomers in the Aβ preparation to the immunological reagent. In various embodiments, the Aβ oligomers comprise Aβ dimmers, Aβ trimers, or both Aβ dimers and Aβ trimers.

It has been discovered that immunological reagents which exhibit greater binding, (e.g., bind preferentially or bind with greater affinity) to one or more Aβ oligomers in a synthetic Aβ preparation as compared to Aβ monomers in the Aβ preparation also produce an improvement in cognition in Tg2576 transgenic mice, as determined by a contextual fear conditioning (CFC) assay. Such CFC assays are discussed further herein and in copending U.S. Patent Application Ser. No. 60/636,842, filed Dec. 15, 2004, U.S. Ser. No. 60/637,253, filed Dec. 16, 2004, and U.S. Ser. No. 60/736,119, filed on Nov. 10, 2005, the entire contents of which are hereby incorporated by reference. The CFC assay evaluates changes in cognition of an animal (typically a mouse or rat) upon treatment with a potential therapeutic compound. Accordingly, the CFC assay provides a direct method for determining the therapeutic effect of agents for preventing or treating cognitive disease, and in particular, a disease or disorder affecting one or more regions of the brains, e.g., the hippocampus, subiculum, cingulated cortex, prefrontal cortex, perirhinal cortex, sensory cortex, and medial temporal lobe. The discovery that the methods of the present invention are predictive of, or strongly correlative with, the results of the CFC assays suggests that the methods of the present invention can provide a supplement to, or alternatively replace, the inherently more time consuming CFC assay (because, e.g., it is an animal study) as a method for identifying immunological reagents having therapeutic efficacy for the treatment of one or more amyloidogenic disorders, or combination of amyloidogenic disorders.

Accordingly, in various aspects, the present invention provides methods for identifying an immunological reagent having therapeutic efficacy by contacting an Aβ preparation with a test immunological reagent (the Aβ preparation comprising Aβ monomers and one or more Aβ oligomers), and determining an increased binding of the test immunological reagent to the one or more Aβ oligomers as compared to the Aβ monomers, such that an immunological reagent having therapeutic efficacy is identified. In various embodiments, the test immunological reagent comprises an Aβ antibody.

In various aspects, the present invention provides methods for identifying an immunological reagent having therapeutic efficacy that contact an Aβ preparation with a test immunological reagent (the Aβ preparation comprising Aβ monomers and one or more Aβ oligomers) and determine the amount of Aβ monomers and one or more Aβ oligomers bound to the test immunological reagent. The amount of Aβ monomers is compared to the amount of one or more Aβ oligomers bound to the test immunological reagent and an increased amount of one or more oligomers bound to the test immunological agent relative to the amount of Aβ monomers bound identifies the immunological reagent as having therapeutic efficacy.

In various aspects, the present invention provides methods for predicting the therapeutic efficacy of an immunological reagent for the treatment of an amyloidogenic disorder by comparing the amount of Aβ monomer in an Aβ preparation which binds to the immunological reagent to an amount of one or more Aβ oligomers in the Aβ preparation which bind to the immunological reagent to determine a relative bound amount, and predicting the efficacy of the immunological reagent for the treatment of an amyloidogenic disorder based at least on the relative bound amount.

In various aspects, the present invention provides methods for predicting the ability of an immunological reagent to neutralize one or more neuroactive forms of Aβ (also referred to herein as “neuroactive Aβ species” or “NAβS”). In various aspects, the present invention features methods for identifying an immunological reagent that can neutralize one or more neuroactive forms of Aβ. Accordingly, in various aspects, the present invention provides methods for identifying an immunological reagent that can neutralize one or more neuroactive Aβ species by contacting an Aβ preparation with a test immunological reagent, wherein the Aβ preparation comprises Aβ monomers and one or more Aβ oligomers, determining the amount of Aβ monomers and one or more Aβ oligomers bound to the test immunological reagent, and comparing the amount of Aβ monomers and one or more Aβ oligomers bound to the test immunological reagent, where an increased amount of one or more oligomers bound to the test immunological agent relative to the amount of Aβ monomers bound identifies the immunological reagent as having the ability to neutralize one or more neuroactive Aβ species.

The methods are based, at least in part, on a comparison of an amount of one or more Aβ oligomers in an Aβ preparation which bind to an immunological reagent as compared to the amount of Aβ monomers in the Aβ preparation which bind to the immunological reagent. The comparison of these binding amounts is used, at least in part, to identify an immunological reagent as having (or not having) the ability to neutralize one or more neuroactive forms of Aβ. In various embodiments, the methods predict the ability of an immunological reagent to neutralize one or more neuroactive forms of one or more Aβ oligomers. Exemplary neuroactive Aβ species include soluble Aβ species.

In various embodiments, an immunological reagent to be tested (a test immunological reagent) for therapeutic efficacy, neutralization of one or more neuroactive forms of Aβ, or both, is contacted with an Aβ preparation. The amount of Aβ monomers in the preparation which bind to the test immunological reagent relative to the amount of one or more of Aβ dimers, trimers, tetramers, pentamers, and/or higher order oligomers in the preparation, which bind to the test immunological reagent is then used, at least in part, to determine whether to identify the test immunological reagent (e.g., an Aβ antibody) as having therapeutic efficacy for the treatment of one or more amyloidogenic disorders, or combination of amyloidogenic disorders.

In various embodiments, the amyloidogenic disorder is Alzheimer's disease. In various embodiments, the amyloidogenic disorder is one or more of systemic amyloidosis, Alzheimer's disease, cerebral amyloid angiopathy, mature onset diabetes, Parkinson's disease, Huntington's disease, fronto-temporal dementia, and the prion-related transmissible spongiform encephalopathies (kuru and Creutzfeldt-Jacob disease in humans and scrapie and BSE in sheep and cattle, respectively), and combinations thereof.

The Aβ preparation with which the test immunological reagent is contacted can be derived from a variety of sources, for example, tissues, cell lines, synthesis, etc. that can provide an Aβ preparation with both Aβ monomers and one or more Aβ oligomers, that is substantially free of fibrils. In various embodiments, the Aβ preparation comprises synthetically-prepared Aβ substantially free of fibrils which is treated with a crosslinking reagent to, for example, stabilize the resultant Aβ preparation. A variety of crosslinking reagents can be used including, but not limited to, amine-amine linkers, thiol-thiol linkers, alcohol-alcohol linkers, carboxylic acid-carboxylic acid linkers, and aryl-aryl linkers (such as, for example, peroxynitrite, and bis-diazobenzidine (BDB)). When using thio linkers, it should be noted that naturally-occurring Aβ42 does not contain cysteine residues. Accordingly, cysteine residues can be engineered at residues unimportant for Aβ function to provide thiol groups for crosslinking. Crosslinking reagents can also be used that link dissimilar functional groups, such as, for example, amine-thiol linkers (e.g., m-maleimidobenzoyl-N-hydrosuccinamide (MBS)), amine-carboxylic acid linkers, amine-carbonyl linkers, thiol-alcohol linkers, thiol-carbonyl linkers, and thiol-carboxylic acid linkers.

In various embodiments, an immunological reagent is identified as having therapeutic efficacy for the treatment of one or more amyloidogenic disorders, an ability to neutralize one or more neuroactive forms of Aβ, or both, when the binding of one or more Aβ oligomers in the Aβ preparation to the immunological reagent is greater than the binding of Aβ monomers in the Aβ preparation to the immunological reagent. For example, in one embodiment, an immunological reagent is identified as having therapeutic efficacy for the treatment of one or more amyloidogenic disorders, an ability to neutralize one or more neuroactive forms of Aβ, or both, when the binding of Aβ dimers to the immunological reagent is greater than the binding of Aβ monomers to the immunological reagent. In another embodiment, an immunological reagent is identified as having therapeutic efficacy for the treatment of one or more amyloidogenic disorders, an ability to neutralize one or more neuroactive forms of Aβ, or both, when the binding of Aβ trimers to the immunological reagent is greater than the binding of Aβ monomers to the immunological reagent. In yet another embodiment, an immunological reagent is identified as having therapeutic efficacy for the treatment of one or more amyloidogenic disorders, an ability to neutralize one or more neuroactive forms of Aβ, or both, when the binding of both Aβ dimers and one or more higher order oligomers (i.e., trimers or greater), to the immunological reagent is greater than the binding of Aβ monomers to the immunological reagent.

In various embodiments, an immunological reagent is identified as having therapeutic efficacy for the treatment of one or more amyloidogenic disorders, an ability to neutralize one or more neuroactive forms of Aβ, or both, when: the amount of Aβ monomer is less than the amount of one or more Aβ oligomers; the amount of Aβ monomer is less than the amount of Aβ dimmers; the amount of Aβ monomer is less than the amount of Aβ trimers; or one or more combinations thereof

In various embodiments, an immunological reagent is identified as having therapeutic efficacy for the treatment of one or more amyloidogenic disorders, an ability to neutralize one or more neuroactive forms of Aβ, or both, when the increased binding of one or more Aβ oligomers in the Aβ preparation to the test immunological reagent compared to that for Aβ monomers in the Aβ preparation, is an increased binding as compared to that for a control reagent contacted with the Aβ preparation. In various embodiments, an immunological reagent is identified as not having therapeutic efficacy or an ability to neutralize one or more neuroactive forms of Aβ, or both, when the binding of one or more Aβ oligomers in the Aβ preparation to the immunological reagent as compared to that for Aβ monomers in the Aβ preparation, is less than the corresponding binding of the corresponding Aβ species to a control reagent.

In various embodiments, the amount of Aβ monomers in the Aβ preparation which bind to the control reagent is substantially equal to the amount of one or more Aβ oligomers in the Aβ preparation which bind to the control reagent. In various embodiments, the amount of Aβ monomers in the Aβ preparation which bind to the control reagent is greater than the amount of one or more Aβ oligomers in the Aβ preparation which bind to the control reagent. In various embodiments, the amount of Aβ monomers in the Aβ preparation which bind to the control reagent is less than the amount of one or more Aβ oligomers in the Aβ preparation which bind to the control reagent.

In other embodiments, an immnunological reagent is identified as having therapeutic efficacy when the affinity of the immunological reagent for one or more Aβ oligomers as compared to Aβ monomers is increased as compared to the affinities of a control reagent. A control reagent, for example, can exhibit a substantially equal affinity for Aβ monomers and one or more Aβ oligomers or a greater affinity for Aβ monomers as compared to Aβ oligomers. The one or more Aβ oligomers can include, for example, one or more of Aβ dimers, Aβ trimers, Aβ tetramers.

In various embodiments, an immunological reagent is identified as having therapeutic efficacy when the ratio of the Aβ monomer affinity to an Aβ oligomer (or combination of oligomers) affinity for the immunological reagent is lower than the corresponding affinity ratio for a control reagent.

In the various aspects of the invention, an increased or greater amount of binding can be determined by a comparison of the amount of the Aβ monomers in the Aβ preparation which bind to the immunological reagent to an amount of one or more Aβ oligomers in the Aβ preparation which bind to the immunological reagent. These amounts can be qualitative, quantitative, or combination of both.

In various embodiments, the amount of Aβ monomers and one or more Aβ oligomer species in an Aβ preparation which bind to a test immunological reagent is assessed using immunoprecipitation to precipitate from the Aβ preparation the Aβ monomers and one or more Aβ oligomer species bound to the test immunological reagent. The amount of Aβ monomer precipitate and the amount of precipitate for one or more Aβ oligomer species is then compared to predict the efficacy of the immunological reagent for the treatment of one or more amyloidogenic disorders, an ability to neutralize one or more neuroactive forms of Aβ, or both. In various embodiments, the amount of Aβ monomers and one or more Aβ oligomer species in an Aβ preparation which bind to a test immunological reagent is assessed using immunoprecipitation of the Aβ monomers and one or more Aβ oligomer from the Aβ preparation followed by an electrophoretic separation on the immunoprecipitate.

In various embodiments, the determination of an increased binding comprises immunodetection of the immunoprecipitated reagent. In various embodiments, immunodetection is achieved using an antibody which detects, labeled, unlabeld, or both labeled and unlabeled, Aβ monomers and Aβ oligomers. A wide variety of labels can be used including, but not limited to, fluorescent labels, radioactive labels, paramagnetic labels, and combinations thereof.

In various embodiments, an immunological reagent is identified as having therapeutic efficacy for the treatment of one or more amyloidogenic disorders, an ability to neutralize one or more neuroactive forms of Aβ, or both, when: the amount of Aβ monomer is low relative to the amount of Aβ oligomers, as compared to corresponding relative amounts of Aβ monomers to Aβ oligomers precipitated by a control reagent contacted with the Aβ preparation; the amount of Aβ monomer is high relative to the amount of Aβ oligomers precipitated by the control reagent; or combinations thereof. The one or more Aβ oligomers can include, for example, one or more of Aβ dimers, Aβ trimers, Aβ tetramers, and combinations thereof.

Accordingly, in various aspects, the present invention provides methods for identifying an immunological reagent having therapeutic efficacy for the treatment of an amyloidogenic disorder by precipitating at least a portion of an Aβ preparation with an immunological reagent, the Aβ preparation comprising Aβ monomers and one or more Aβ oligomers, comparing the amount of precipitated Aβ monomer to the amount of precipitated Aβ oligomers, and identifying the immunological reagent as having therapeutic efficacy for the treatment of the amyloidogenic disorder based at least on the amount of Aβ monomer relative to the amount of Aβ oligomers.

In various aspects, the present invention features methods for predicating the results, corroborating the results, or both, of an animal assay for identifying immunological reagents having therapeutic efficacy for the treatment of one or more amyloidogenic disorders (e.g., Alzheimer's disease), or combination of amyloidogenic disorders. These animal assays are based, at least in part, on comparing cognition, as determined from a contextual fear conditioning study of an animal after administration of a test immunological reagent to the animal, as compared to a suitable control.

In various aspects, the present invention features methods for for identifying an immunological reagent having the ability to effect a rapid improvement in cognition in an animal by contacting an Aβ preparation with a test immunological reagent, wherein the Aβ preparation comprises Aβ monomers and one or more Aβ oligomers, determining an increased binding of the test immunological reagent to the Aβ oligomers as compared to the Aβ monomers, such that an immunological reagent having the ability to effect a rapid improvement in cognition in an animal is identified; and, in various embodiments, confirming in a test animal a rapid improvement in cognition. Where, for example, the animal is a human and/or the test animal is an animal model for Alzheimer's Disease tested in contextual fear conditioning (CFC).

The present invention also features, in various aspects, kits for use in performing one or more of the methods of the present invention. In various II embodiments, a kit comprises one or more Aβ antibodies and reagents for the preparation of an Aβ preparation.

### BRIEF DESCRIPTION OF THE DRAWINGS

FIG. 1 depicts an oligomer profile of a 7PA2 versus Chinese Hamster Ovary (CHO) cell conditioned medium (CM) immunoprecipitated with various Aβ antibodies and imaged with 6E 10 (Aβ epitope 6-10). The right panel depicts CM immunoprecipitated with the monoclonal antibody (mAb) 21F12 or the polyclonal Ab R1282. M, D and T indicate monomeric, dimeric and trimeric Aβ species, respectively. The left panel depicts CM immunoprecipitated with the mAbs 21F12, 3D6, 12A11 or 2C1, or the pAb R1282. Molecular weight markers are indicated.

FIG. 2 depicts an Aβ profile (imaged with 6E 10) of 7PA2 or CHO CM immunoprecipitated with 21F12 or RI 282 compared with various amounts of monomeric Aβ₁₋₄₂ Molecular weight markers are indicated. The approximate positions of Aβ₁₋₄₂ monomer, dimer and trimer bands are indicated on the right-hand side of the figure.

FIG. 3 depicts a Western blot of a HFIP-solubilized Aβ₁₋₄₂ preparation subject to various treatment conditions (water (H₂O), sodium hydroxide (NaOH) or peroxynitrite) at various incubation times after treatment.

FIG. 4 depicts a Western blot of a DMSO-sloublized AP₁₋₄₂ preparation subject to various treatment conditions (water (H₂O), sodium hydroxide (NaOH) or peroxynitrite) at various incubation times after treatment.

FIG. 5 depicts a Western blot of immunoprecipitates of a peroxynitrite treated Aβ preparation precipitated with various Aβ antibodies (3D6, 6C6, 12B4, 15C11, 3A3, 5A11, 6H9, and 266) and imaged with 3D6. The approximate positions of Aβ₁₋₄₂ monomer, dimer, trimer and tetramer bands are indicated on the left-hand side of the figure.

FIG. 6 depicts a Western blot of immunoprecipitates of peroxynitrite treated Aβ preparation precipitated with various Aβ antibodies (3D6, 6C6, 12A11, 12B4, 3A3, 266, 9G8, 15C11, and 6H9) and imaged with 3D6. Annotation is the same as for FIG. 5.

FIG. 7 depicts a Western blot of immunoprecipitates of peroxynitrite treated Aβ preparation precipitated with various Aβ antibodies (3D6, 6C6, 12A11, 12B4, 10D5, 3A3, 266 and 6H9) and imaged with 3D6. Annotation is the same as for FIG. 5.

FIG. 8 depicts a Western blot of immunoprecipitates of peroxynitrite treated Aβ preparation precipitated with various Aβ antibodies (3A3, lane 2, and 6H9, lane 3) compared with Aβ preparation alone (lane 1) and a mock 6H9 immunoprecipitation reaction with no Aβ ₁₋₄₂ oligomers.

### DETAILED DESCRIPTION OF THE INVENTION

The present invention features, in various aspects, methods for identifying immunological reagents having therapeutic efficacy for the treatment of Alzheimer's disease or other amyloidogenic disorders. In various aspects, the present invention provides methods for predicting the therapeutic efficacy of an immunological reagent for the treatment of an amyloidogenic disorder by comparing the amount of Aβ monomer in an Aβ preparation which binds to the immunological reagent to an amount of one or more Aβ oligomers in the Aβ preparation which bind to the immunological reagent to predict the efficacy of the immunological reagent for the treatment of an amyloidogenic disorder. The comparison of amounts can be a qualitative comparison, a quantitative comparison, or combination of both. The amount of an Aβ species bound to, or which binds to, an immunological reagent can be a qualitative determination, a quantitative determination, or a combination of both.

In other aspects, the assays of the invention feature comparison of an immunological reagent's affinity for one or more Aβ oligomers in an Aβ preparation as compared to the immunological reagent's affinity for Aβ monomers in the Aβ preparation. The comparison of these affinities leads to identifying an immunological reagent as having, or not having, therapeutic efficacy for the treatment of one or more amyloidogenic disorders. In particular, the invention features identification of Aβ antibodies having therapeutic efficacy for the treatment of Alzheimer's disease (AD).

In various aspects, the present invention features methods for predicting the ability of an immunological reagent to neutralize one or more neuroactive soluble forms of Aβ. The methods are based, at least in part, on a comparison of an immunological reagent's affinity for one or more Aβ oligomers as compared to the immunological reagent's affinity for Aβ monomers. The comparison of these affinities leads to identifying an immunological reagent as having (or not having) the ability to neutralize one or more neuroactive soluble forms of Aβ. For example, therapeutic approaches focused solely on fibril destabilization may have the undesirable side effect of increasing a soluble pool or neuroactive Aβ oligomers and protofibrils. In various embodiments of the present invention, the methods for predicting the ability of an immunological reagent to neutralize one or more neuroactive soluble forms of Aβ can be used to develop a therapeutic approach that includes fibril destabilization as well as decreasing (e.g., by neutralization) soluble neuroactive Aβ species. In various embodiments, the methods predict the ability of an immunological reagent to decrease one or more neuroactive soluble forms of one or more Aβ dimers, trimer, tetramers, pentamers, higher ordered oligomers, or combinations thereof.

In various embodiments, an immunological reagent to be tested (a test immunological reagent) for therapeutic efficacy, neutralization of one or more neuroactive forms of Aβ, or both, is contacted with an Aβ preparation. The Aβ preparation can be derived from a variety of sources, for example, tissues, cell lines, synthesis, etc. that can provide an Aβ preparation with both Aβ monomers and one or more Aβ oligomers. In various embodiments, the Aβ preparation comprises a synthetically prepared preparation substantially free of fibrils which is treated with peroxynitrite. The affinity of the test immunological reagent for Aβ monomers in the Aβ preparation relative to the test immunological reagent's affinity for one or more Aβ oligomers (e.g., dimers, trimers and tetramers) in the Aβ preparation, is assessed and used, at least in part, to determine whether the test immunological reagent has therapeutic efficacy for the treatment of one or more amyloidogenic disorders, or combination of amyloidogenic disorders.

In various embodiments, an immunological reagent is identified as having therapeutic efficacy when the affinity of the immunological reagent for one or more Aβ oligomers compared to Aβ monomers is increased as compared to a control reagent. A control reagent, for example, can exhibit a substantially equal affinity for Aβ monomers and one or more Aβ oligomers. The one or more Aβ oligomers can include, for example, one or more of Aβ dimers, Aβ trimers, Aβ tetramers.

In various embodiments, an immunological reagent is identified as having therapeutic efficacy when the ratio of the Aβ monomer affinity to Aβ oligomer affinity (or combination of oligomer affinities) for the immunological reagent is lower than the corresponding affinity ratio for a control reagent.

Labels can be used to assess the affinity of an immunological reagent for Aβ monomers, Aβ oligomers, or both. In various embodiments, a primary reagent with affinity for Aβ is unlabelled and a secondary labeling agent is used to bind to the primary reagent. Suitable labels include, but are not limited to, fluorescent labels, paramagnetic labels, radioactive labels, and combinations thereof.

The present invention is directed inter alia to identifying immunological reagents (e.g., humanized immunoglobulins to specific epitopes within Aβ) having therapeutic efficacy for the treatment of Alzheimer's and other amyloidogenic diseases. The term “treatment” as used herein, is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A treatment having a therapeutic effect can be one wherein a beneficial therapeutic response is generated in a patient (e.g., induction of phagocytosis of Aβ, reduction of plaque burden, inhibition of plaque formation, reduction of neuritic dystrophy, improving cognitive function, and/or reversing, treating or preventing cognitive decline), for the prophylaxis or treatment of an amyloidogenic disease.

Immunological reagents of the invention are typically substantially pure from undesired contaminants. This means that a reagent is typically at least about 50% w/w (weight/weight) purity, as well as being substantially free from interfering proteins and contaminants. Sometimes the reagents are at least about 80% w/w and, more preferably at least 90 or about 95% w/w purity. However, using conventional protein purification techniques, homogeneous peptides of at least 99% w/w can be obtained.

Immunological reagents of the invention include antibodies that specifically bind to Aβ, for example, soluble Aβ (e.g., soluble oligomeric Aβ and/or soluble monomeric Aβ), aggregated Aβ, protofibrillar Aβ, fibrillar Aβ, Aβ in amyloid plaques and the like. Such antibodies can be monoclonal or polyclonal. Some such antibodies bind specifically to aggregated forms of Aβ without binding to soluble forms. Some bind specifically to soluble forms without binding to aggregated forms. Some bind to both aggregated and soluble forms of Aβ. Some such antibodies bind to a naturally occurring short form of Aβ (i.e., Aβ39, 40 or 41) without binding to a naturally occurring long form of Aβ (i.e., Aβ42 and Aβ43). Some antibodies bind to a long form of Aβ without binding to a short form. Some antibodies bind to Aβ without binding to full-length amyloid precursor protein. Preferred antibodies bind to Aβ (for example, Aβ dimers, trimers, tetramers, pentamers, etc.) with a binding affinity greater than (or equal to) about 10⁶, 10⁷, 10⁸, 10⁹, or 10¹⁰ M⁻¹ (including affinities intermediate of these values).

Polyclonal sera typically contain mixed populations of antibodies binding to several epitopes along the length of Aβ. However, polyclonal sera can be specific to a particular segment of Aβ, such as Aβ1-10. Monoclonal antibodies bind to a specific epitope within Aβ that can be a conformational or nonconformational epitope. Prophylactic and therapeutic efficacy of antibodies can be tested using the transgenic animal model procedures described in the Examples. Exemplary monoclonal antibodies bind to an epitope within residues 1-10 or 1-12 of Aβ (with the first N terminal residue of natural Aβ designated 1). Some monoclonal antibodies bind to an epitope within amino acids 1-5, and some to an epitope within 5-10. Some antibodies bind to epitopes within amino acids 1-3, 1-4, 1-5, 1-6, 1-7 or 3-7. Some antibodies bind to an epitope starting at residues 1-3 and ending at residues 7-11 of Aβ. Other exemplary antibodies include those binding to epitopes with residues 10-15, 15-20, 25-30, 10-20, 20-30, or 10-25 of Aβ. Antibodies can be screened for activity in art-recognized mouse models for other biological activities including, but not limited to, the ability to reduce plaque burden and/or resolve the neuritic burden associated with Alzheimer's disease. For example, it has been found that certain antibodies to epitopes within residues 1-10 (e.g., within residues 1-5 or 3-6 or 3-7) have the ability to reduce plaque burden and neuritic pathology whereas certain antibodies to epitopes within residues 10-18, 16-24, 18-21 and 33-42 lack such activities (e.g., lack the ability to reduce plaque burden and/or resolve neuritic pathology). In some methods, multiple monoclonal antibodies having binding specificities to different epitopes are used. Such antibodies can be administered sequentially or simultaneously. Antibodies to amyloid components other than Aβ can also be used (e.g., administered or co-administered).

When an antibody is said to bind to an epitope within specified residues, such as Aβ 1-5 for example, what is meant is that the antibody specifically binds to a polypeptide containing the specified residues (i.e., Aβ1-5 in this an example). Such an antibody does not necessarily contact every residue within Aβ1-5. Nor does every single amino acid substitution or deletion with in Aβ1-5 necessarily significantly affect binding affinity. Epitope specificity of an antibody can be determined, for example, by forming a phage display library in which different members display different subsequences of Aβ. The phage display library is then selected for members specifically binding to an antibody under test. A family of sequences is isolated. Typically, such a family contains a common core sequence, and varying lengths of flanking sequences in different members. The shortest core sequence showing specific binding to the antibody defines the epitope bound by the antibody.

Epitope specificity of an antibody can also be determined, for example, by replacement NET (rNET) analysis. The rNET epitope map assay provides information about the contribution of individual residues within the epitope to the overall binding activity of the antibody. rNET analysis uses synthesized systematic single substituted peptide analogs. Binding of an antibody being tested is determined against native peptide (native antigen) and against 19 alternative “single substituted” peptides, each peptide being substituted at a first position with one of 19 non-native amino acids for that position. A profile is generated reflecting the effect of substitution at that position with the various non-native residues. Profiles are likewise generated at successive positions along the antigenic peptide. The combined profile, or epitope map, (reflecting substitution at each position with all 19 non-native residues) can then be compared to a map similarly generated for a second antibody. Substantially similar or identical maps indicate that antibodies being compared have the same or similar epitope specificity.

Antibodies can also be tested for epitope specificity in a competition assay with an antibody whose epitope specificity has already been determined. For example, antibodies that compete with the 3D6 antibody for binding to Aβ bind to the same or similar epitope as 3D6, i.e., within residues Aβ1-5. Likewise antibodies that compete with the 10D5 antibody bind to the same or similar epitope, i.e., within residues Aβ3-7. Screening antibodies for epitope specificity is a useful predictor of therapeutic efficacy. For example, an antibody determined to bind to an epitope within residues 1-7 of Aβ is likely to be effective in preventing and treating Alzheimer's disease according to the methodologies of the present invention.

Monoclonal or polyclonal antibodies that specifically bind to a preferred epitope of Aβ without binding to other regions of Aβ have a number of advantages relative to monoclonal antibodies binding to other regions or polyclonal sera to intact Aβ. First, for equal mass dosages, dosages of antibodies that specifically bind to preferred epitopes contain a higher molar dosage of antibodies effective in achieving a desired therapeutic result, e.g., neutralizing or clearing Aβ. Second, antibodies specifically binding to preferred epitopes can induce a response against Aβ without inducing a similar response against intact APP polypeptide, thereby reducing the potential side effects.

Immunological reagents to be tested utilizing the methodologies of the instant invention include monoclonal and/or polyclonal antibodies, as described above, in particular, monoclonal and/or polyclonal antibodies that bind to Aβ. Humanized, chimeric and variant antibodies (e.g., Fc variants, affinity matured variants) are also exemplary immunological reagents to be tested utilizing the methodologies of the invention. Likewise, antigen-binding (e.g., Aβ-binding) antibody chains, domains, regions, fragments and the like are exemplary immunological reagents to be tested utilizing the methodologies of the invention. Other aspect of the invention feature testing immunological reagents isolated from certain subjects for their Aβ binding abilities (e.g. ,ability to bind to soluble, oligomeric Aβ as compared to monomeric Aβ). In exemplary embodiments, the immunological reagent is a serum sample from the subject. In certain embodiments, the immunological reagent is a serum sample from a human subject. For example, human serum samples can be assayed according to the methodologies of the instant invention to determine whether a subject (e.g., a patient) is producing desired titers of antibodies (e.g., Aβ antibodies) in response to administration of an antigen or vaccine. In other exemplary embodiments, the immunological reagent is a serum sample from an animal subject (e.g., a pre-clinical animal model). For example, animal serum samples can assayed according to the methodologies of the instant invention to determine whether a test immunogen elicits a desired immunologic responses (e.g., production of antibodies binding to oligomeric Aβ species). In such applications, the methods of the invention are useful to optimize immunogens in a pre-clinical setting, for example, to identify candidate immunogens.

In various aspects, the present invention features animal assays for validating an immunological reagent's therapeutic efficacy for the treatment of one or more amyloidogenic disorders (e.g., Alzheimer's disease), or combination of amyloidogenic disorders. The assays are based, at least in part, on comparing cognition, as determined from a contextual fear conditioning study of the animal, before and after administration of a test immunological reagent to the animal.

Prior to further describing the invention, it may be helpful to provide an understanding thereof to set forth definitions of certain terms to be used herein.

The term “immunological reagent” or “immunoreagent” (used interchangeably herein) refers to an agent that comprises or consists of one or more immunogens, immunoglobulins, antibodies, or functional or antigen binding fragments thereof, as defined herein, or combinations thereof. Accordingly, as used herein, the term “immunological reagent” or “immunoreagent” also includes nucleic acids encoding antibodies and their component chains used for passive immunization. Such nucleic acids can be DNA or RNA. A nucleic acid segment encoding an immunogen is typically linked to regulatory elements, such as a promoter and enhancer, that allow expression of the DNA segment in the intended target cells of a patient.

The term “crosslinking reagent” refers to one or more reagents that can be used to crosslink polypeptides. The term crosslinking reagent includes, for example, biaryl crosslinking reagents (e.g., dityrosine crosslinking reagents, ditryptophan crosslinking reagents, etc.), diamine crosslinking reagents, and disulphide crosslinking reagents. The term crosslinking reagent includes photoactive crosslinking reagents which require light (typically within a narrow band of wavelengths) to initiate the crosslinking reaction. The term crosslinking reagent also includes homobifunctional crosslinking reagents (e.g., amine-amine linkers, thiol-thiol linkers, alcohol-alcohol linkers, etc.) and heterobifunctional crosslinking reagents (e.g., amine-thiol linkers, amine-carboxylic acid linkers, amine-carbonyl linkers, thiol-alcohol linkers, thiol-carbonyl linkers, thiol carboxylic acid linkers, etc.).

The term “immunoglobulin” or “antibody” (used interchangeably herein) refers to an antigen-binding protein having a basic four-polypeptide chain structure consisting of two heavy and two light chains, said chains being stabilized, for example, by interchain disulfide bonds, which has the ability to specifically bind antigen. Both heavy and light chains are folded into domains. The term “domain” refers to a globular region of a heavy or light chain polypeptide comprising peptide loops (e.g., comprising 3 to 4 peptide loops) stabilized, for example, by β-pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. “Constant” domains on the light chain are referred to interchangeably as “light chain constant regions”, “light chain constant domains”, “CL” regions or “CL” domains). “Constant” domains on the heavy chain are referred to interchangeably as “heavy chain constant regions”, “heavy chain constant domains”, “CH” regions or “CH” domains). “Variable” domains on the light chain are referred to interchangeably as “light chain variable regions”, “light chain variable domains”, “VL” regions or “VL” domains). “Variable” domains on the heavy chain are referred to interchangeably as “heavy chain constant regions”, “heavy chain constant domains”, “CH” regions or “CH” domains).

The term “region” refers to a part or portion of an antibody chain and includes constant or variable domains as defined herein, as well as more discrete parts or portions of said domains. For example, light chain variable domains or regions include “complementarity determining regions” or “CDRs” interspersed among “framework regions” or “FRs”, as defined herein.

Immunoglobulins or antibodies can exist in monomeric or polymeric form. The term “antigen-binding fragment” refers to a polypeptide fragment of an immunoglobulin or antibody binds antigen or competes with intact antibody (i.e., with the intact antibody from which they were derived) for antigen binding (i.e., specific binding). The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “immunoglobulin” or “antibody” includes humanized immunoglobulins or antibodies, chimeric immunoglobulins, and immunoglobulins having altered effector function, such as the ability to bind effector molecules, for example, complement or a receptor on an effector cell.

“Specific binding” of an antibody means that the antibody exhibits appreciable affinity for antigen or a preferred epitope and, preferably, does not exhibit significant crossreactivity. “Appreciable” or preferred binding include binding with an affinity of at least 10⁶, 10⁷, 10⁸, 10⁹ M⁻¹, or 10¹⁰M⁻¹. Affinities greater than 10⁷M⁻¹, preferably greater than 10⁸M⁻¹ are more preferred. Values intermediate of those set forth herein are also intended to be within the scope of the present invention and a preferred binding affinity can be indicated as a range of affinities, for example, 10⁶ to 10¹⁰M⁻¹, preferably 10⁷ to 10¹⁰M⁻¹, more preferably 10⁸ to 10¹⁰M⁻¹. An antibody that “does not exhibit significant crossreactivity” is one that will not appreciably bind to an undesirable entity (e.g., an undesirable proteinaceous entity). For example, an antibody that specifically binds to Aβ will appreciably bind Aβ but will not significantly react with non-Aβ proteins or peptides (e.g., non-Aβ proteins or peptides included in plaques). Likewise, an antibody that specifically binds to Aβ dimers, trimers, tetramers, etc. will appreciably bind said Aβ dimers, trimers, tetramers, etc., respectively, but will not significantly react with, for example, Aβ monomers. An antibody specific for a preferred epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

Binding fragments are produced by recombinant DNA techniques, or by enzymatic or chemical cleavage of intact immunoglobulins. Binding fragments include Fab, Fab′, F(ab′)₂, Fabc, Fv, single chains, and single-chain antibodies. Other than “bispecific” or “bifunctional” immunoglobulins or antibodies, an immunoglobulin or antibody is understood to have each of its binding sites identical. A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

The term “humanized immunoglobulin” or “humanized antibody” refers to an immunoglobulin or antibody that includes at least one humanized immunoglobulin or antibody chain (i e., at least one humanized light or heavy chain). The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and complementarity determining regions (CDRs) (e.g., at least one CDR, preferably two CDRs, more preferably three CDRs) substantially from a non-human immunoglobulin or antibody, and further includes constant regions (e.g., at least one constant region or portion thereof, in the case of a light chain, and preferably three constant regions in the case of a heavy chain). The term “humanized variable region” (e.g., “humanized light chain variable region” or “humanized heavy chain variable region”) refers to a variable region that includes a variable framework region substantially from a human immunoglobulin or antibody and complementarity determining regions (CDRs) substantially from a non-human immunoglobulin or antibody.

The phrase “substantially from a human acceptor immunoglobulin” means that the majority or key framework residues are from the human acceptor sequence, allowing however, for substitution of residues at certain positions with residues selected to improve activity of the humanized immunoglobulin (e.g., alter activity such that it more closely mimics the activity of the donor immunoglobulin) or selected to decrease the immunogenicity of the humanized immunoglobulin.

The phrase “substantially from a human immunoglobulin or antibody” or “substantially human” means that, when aligned to a human immunoglobulin or antibody amino sequence for comparison purposes, the region shares at least 80-90%, preferably 90-95%, more preferably 95-99% identity (i.e., local sequence identity) with the human framework or constant region sequence, allowing, for example, for conservative substitutions, consensus sequence substitutions, germline substitutions, backmutations, and the like. The introduction of conservative substitutions, consensus sequence substitutions, germline substitutions, backmutations, and the like, is often referred to as “optimization” of a humanized antibody or chain. The phrase “substantially from a non-human immunoglobulin or antibody” or “substantially non-human” means having an immunoglobulin or antibody sequence at least 80-95%, preferably 90-95%, more preferably, 96%, 97%, 98%, or 99% identical to that of a non-human organism, e.g., a non-human mammal.

Accordingly, all regions or residues of a humanized immunoglobulin or antibody, or of a humanized immunoglobulin or antibody chain, except possibly the CDRs, are substantially identical to the corresponding regions or residues of one or more native human immunoglobulin sequences. The term “corresponding region” or “corresponding residue” refers to a region or residue on a second amino acid or nucleotide sequence which occupies the same (i.e., equivalent) position as a region or residue on a first amino acid or nucleotide sequence, when the first and second sequences are optimally aligned for comparison purposes.

The terms “humanized immunoglobulin” or “humanized antibody” are not intended to encompass chimeric immunoglobulins or antibodies, as defined infra. Although humanized immunoglobulins or antibodies are chimeric in their construction (i.e., comprise regions from more than one species of protein), they include additional features (i.e., variable regions comprising donor CDR residues and acceptor framework residues) not found in chimeric immunoglobulins or antibodies, as defined herein.

The term “significant identity” means that two polypeptide sequences, when optimally aligned, such as by the programs GAP or BESTFIT using default gap weights, share at least 50-60% sequence identity, preferably 60-70% sequence identity, more preferably 70-80% sequence identity, more preferably at least 80-90% identity, even more preferably at least 90-95% identity, and even more preferably at least 95% sequence identity or more (e.g., 99% sequence identity or more). The term “substantial identity” means that two polypeptide sequences, when optimally aligned, such as by the programs GAP or BESTFIT using default gap weights, share at least 80-90% sequence identity, preferably 90-95% sequence identity, and more preferably at least 95% sequence identity or more (e.g., 99% sequence identity or more). For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. The terms “sequence homology” and “sequence identity” are used interchangeably herein.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols in Molecular Biology). One example of algorithm that is suitable for determining percent sequence identity and sequence similarity is the BLAST algorithm, which is described in Altschul et al., J. Mol. Biol. 215:403 (1990). Software for performing BLAST analyses is publicly available through the National Center for Biotechnology Information (publicly accessible through the National Institutes of Health NCBI internet server). Typically, default program parameters can be used to perform the sequence comparison, although customized parameters can also be used. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

Preferably, residue positions which are not identical differ by conservative amino acid substitutions. For purposes of classifying amino acids substitutions as conservative or nonconservative, amino acids are grouped as follows: Group I (hydrophobic sidechains): leu, met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

Preferably, humanized immunoglobulins or antibodies bind antigen with an affinity that is within a factor of three, four, or five of that of the corresponding non-human antibody. For example, if the nonhuman antibody has a binding affinity of 10⁹ M⁻¹, humanized antibodies will have a binding affinity of at least 3×10⁹ M⁻¹, 4×10⁹ M⁻¹ or 10⁹ M⁻¹. When describing the binding properties of an immunoglobulin or antibody chain, the chain can be described based on its ability to “direct antigen (e.g., Aβ) binding”. A chain is said to “direct antigen binding” when it confers upon an intact immunoglobulin or antibody (or antigen binding fragment thereof) a specific binding property or binding affinity. A mutation (e.g., a backmutation) is said to substantially affect the ability of a heavy or light chain to direct antigen binding if it affects (e.g., decreases) the binding affinity of an intact immunoglobulin or antibody (or antigen binding fragment thereof) comprising said chain by at least an order of magnitude compared to that of the antibody (or antigen binding fragment thereof) comprising an equivalent chain lacking said mutation. A mutation “does not substantially affect (e.g., decrease) the ability of a chain to direct antigen binding” if it affects (e.g., decreases) the binding affinity of an intact immunoglobulin or antibody (or antigen binding fragment thereof) comprising said chain by only a factor of two, three, or four of that of the antibody (or antigen binding fragment thereof) comprising an equivalent chain lacking said mutation.

The term “chimeric immunoglobulin” or antibody refers to an immunoglobulin or antibody whose variable regions derive from a first species and whose constant regions derive from a second species. Chimeric immunoglobulins or antibodies can be constructed, for example by genetic engineering, from immunoglobulin gene segments belonging to different species.

An “antigen” is an entity (e.g., a proteinaceous entity or peptide) to which an antibody specifically binds.

The term “epitope” or “antigenic determinant” refers to a site on an antigen to which an immunoglobulin or antibody (or antigen binding fragment thereof) specifically binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, G. E. Morris, Ed. (1996).

Antibodies that recognize the same epitope can be identified in a simple immunoassay showing the ability of one antibody to block the binding of another antibody to a target antigen, i.e., a competitive binding assay. Competitive binding is determined in an assay in which the immunoglobulin under test inhibits specific binding of a reference antibody to a common antigen, such as Aβ. Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using I-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)). Typically, such an assay involves the use of purified antigen bound to a solid surface or cells bearing either of these, an unlabeled test immunoglobulin and a labeled reference immunoglobulin. Competitive inhibition is measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70%, 70-75% or more.

An epitope is also recognized by immunologic cells, for example, B cells and/or T cells. Cellular recognition of an epitope can be determined by in vitro assays that measure antigen-dependent proliferation, as determined by ³H-thymidine incorporation, by cytokine secretion, by antibody secretion, or by antigen-dependent killing (cytotoxic T lymphocyte assay).

Exemplary epitopes or antigenic determinants can be found within the human amyloid precursor protein (APP), but are preferably found within the Aβ peptide of APP. Multiple isoforms of APP exist, for example APp⁶⁹⁵ APP⁷⁵¹ and APP⁷⁷⁰. Amino acids within APP are assigned numbers according to the sequence of the APP⁷⁷⁰ isoform (see e.g., GenBank Accession No. P05067, also set forth as SEQ ID NO:38). Aβ (also referred to herein as beta amyloid peptide and A-beta) peptide is a −4 kDa internal fragment of 39-43 amino acids of APP (Aβ39, Aβ40, Aβ41, Aβ42 and Aβ43). Aβ40, for example, consists of residues 672-711 of APP and Aβ42 consists of residues 672-713 of APP. As a result of proteolytic processing of APP by different secretase enzymes iv vivo or in situ, Aβ is found in both a “short form”, 40 amino acids in length, and a “long form”, ranging from 42-43 amino acids in length. Exemplary epitopes or antigenic determinants, as described herein, are located within the N-terminus of the Aβ peptide and include residues within amino acids 1-10 or 1-12 of Aβ, preferably from residues 1-3, 4, 5, 6, or 7 of Aβ42 or 3-7 of Aβ42. Additional exemplary epitopes or antigenic determinants include residues 2-4, 5, 6, 7 or 8 of Aβ, residues 3-5, 6, 7, 8 or 9 of Aβ, or residues 4-7, 8, 9 or 10 of Aβ42. Such epitopes can be referred to as N-terminal epitopes. Additional exemplary epitopes or antigenic determinants include residues 19-22, 23 or 24 of Aβ42. Additional exemplary epitopes or antigenic determinants include residues 13-28 of Aβ, preferably residues 16-21, 22, 23 or 24 of Aβ42, or residues 18-21, 19-21, 22, 23 or 24 of Aβ. Such epitopes can be referred to as central epitopes. Additional exemplary epitopes or antigenic determinants include residues 33-40 or 33-42 of Aβ. Such epitopes can be referred to as C-terminal epitopes (i.e., are within about residues 30-40 or 30-42 of Aβ).

The term “amyloidogenic disorder” includes any disease or disorder associated with (or caused by) the formation or deposition of insoluble amyloid fibrils. Exemplary amyloidogenic diseases include, but are not limited to systemic amyloidosis, Alzheimer's disease (AD), cerebral amyloid angiopathy (CAA), mature onset diabetes, Parkinson's disease, Huntington's disease, fronto-temporal dementia, and the prion-related transmissible spongiform encephalopathies (kuru and Creutzfeldt-Jacob disease in humans and scrapie and BSE in sheep and cattle, respectively). Different amyloidogenic diseases are defined or characterized by the nature of the polypeptide component of the fibrils deposited. For example, in subjects or patients having Alzheimer's disease, β-amyloid protein (e.g., wild-type, variant, or truncated β-amyloid protein) is the characterizing polypeptide component of the amyloid deposit. Accordingly, Alzheimer's disease is an example of a “disease characterized by deposits of Aβ” or a “disease associated with deposits of Aβ”, e.g., in the brain of a subject or patient. The terms “P-amyloid protein”, “β-amyloid peptide”, “β-amyloid”, “Aβ” and “Aβ peptide” are used interchangeably herein.

As used herein, the phrase “neuroactive Aβ species” refers to an Aβ species (e.g., an Aβ peptide or form of Aβ peptide) that effects at least one activity or physical characteristic of a neuronal cell. Neuroactive Aβ species effect, for example, the function, biological activity, viability, morphology and/or architecture of a neuronal cell. The effect on neuronal cells can be cellular, for example, effecting the long-term-potentiation (LTP) of a neuronal cell or viability of a neuronal cell (neurotoxicity). The effects of Aβ on neuronal function can also be mediated indirectly, for example by activation of glial cells which in turn affect the neurons (Wang et. al., J. Neurosci., 24:6049 (2004)). Alternatively, the effect can be on an in vivo neuronal system, for example, effecting a behavioral outcome in an appropriate animal test (e.g., a cognitive test). The term “neutralize” as used herein means to make neutral, counteract or make ineffective an activity or effect.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the infection and the general state of the patient's own immune system.

The term “patient” includes human and other mammalian subjects that receive either prophylactic or therapeutic treatment. “Soluble” or “dissociated” Aβ refers to non-aggregating or disaggregated Aβ polypeptide. “Insoluble” Aβ refers to aggregating Aβ polypeptide, for example, Aβ held together by noncovalent bonds. Aβ (e.g., Aβ42) is believed to aggregate, at least in part, due to the presence of hydrophobic residues at the C-terminus of the peptide (part of the transmembrane domain of APP). One method to prepare soluble Aβ is to dissolve lyophilized peptide in neat DMSO with sonication. The resulting solution is centrifuged to remove any insoluble particulates.

The term “effector function” refers to an activity that resides in the Fc region of an antibody (e.g., an IgG antibody) and includes, for example, the ability of the antibody to bind effector molecules such as complement and/or Fc receptors, which can control several immune functions of the antibody such as effector cell activity, lysis, complement-mediated activity, antibody clearance, and antibody half-life.

The term “effector molecule” refers to a molecule that is capable of binding to the Fc region of an antibody (e.g., an IgG antibody) including, but not limited to, a complement protein or a Fc receptor.

The term “effector cell” refers to a cell capable of binding to the Fc portion of an antibody (e.g., an IgG antibody) typically via an Fc receptor expressed on the surface of the effector cell including, but not limited to, lymphocytes, e.g., antigen presenting cells and T cells.

The term “Fc region” refers to a C-terminal region of an IgG antibody, in particular, the C-terminal region of the heavy chain(s) of said IgG antibody. Although the boundaries of the Fc region of an IgG heavy chain can vary slightly, a Fc region is typically defined as spanning from about amino acid residue Cys226 to the carboxyl-terminus of an IgG heavy chain(s).

The term “Kabat numbering” unless otherwise stated, is as taught in Kabat et al. (Sequences of Proteins of Immunological Interest, 5th Ed. Public Health Service, National Institutes of Health, Bethesda, Md. (1991)), expressly incorporated herein by reference. “EU numbering” unless otherwise stated, is also taught in Kabat et al. (Sequences of Proteins of Immunological Interest, 5th Ed. Public Health Service, National Institutes of Health, Bethesda, Md. (1991)) and, for example, refers to the numbering of the residues in heavy chain antibody sequences using the EU index as described therein.

The term “Fc receptor” or “FcR” refers to a receptor that binds to the Fc region of an antibody. Typical Fc receptors which bind to an Fc region of an antibody (e.g., an IgG antibody) include, but are not limited to, receptors of the FcγRI, FcγRII, and FcγRIII subclasses, including allelic variants and alternatively spliced forms of these receptors. Fc receptors are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995).

The term “suitable control” includes any control sample, subject, value, etc. appreciated by the skilled artisan to be appropriate for the parameter being tested. Suitable controls include, for example, samples or subjects having known or predicted characteristics or known or predicted values. Suitable control samples include, but are not limited to buffers, solvents, solids, gasses, particles, media, water, biologicals, empty wells, vessels, etc. Control samples include samples of a like or similar nature to a test agent or sample but having a known or predicted characteristic, e.g., negative or positive control samples. Control subjects include unaffected subjects, unaltered subjects, wild-type subjects, unmanipulated subjects, untreated subjects, and the like. Control values include known or predicted values for a test, test parameter, test condition, etc., such knowledge being based, for example, on past observation or data, and the like. Controls can be physically included in a test of assay in any format. Exemplary controls are positive controls and/or negative controls. Data can be normalized to such controls in certain tests or assays.

I. Aβ Oligomer Preparations

In various aspects of the methods of the present invention, an immunological reagent to be tested for therapeutic efficacy, neutralization of one or more neuroactive forms of Aβ, or both, (a test immunological reagent) is contacted with an Aβ preparation. The methods are based, at least in part, on a comparison of the binding of one or more Aβ oligomers in the Aβ preparation to the test immunological reagent as compared to the binding of Aβ monomers to the test immunological reagent. In other aspects, the methods are based, at least in part, on a comparison of an immunological reagent's affinity for one or more Aβ oligomers in the Aβ preparation as compared to the immunological reagent's affinity for Aβ monomers in the Aβ preparation. The comparison of these affinities leads to identifying an immunological reagent as having (or not having) therapeutic efficacy for the treatment of one or more amyloidogenic disorders.

The Aβ monomers and Aβ oligomers present in an Aβ preparation can be determined or visualized by a wide variety of techniques. For example, in various embodiments, immunoprecipitation and Western blot analysis can be used to visualize the Aβ monomers and oligomers present in the Aβ preparation. Aβ oligomers have molecular weights of, for example, about 8 kDa, about 12 kDa, about 16 kDa or about 20 kDa for dimers, trimers, tetramers or pentamers, respectively. Monomers and oligomers can consist of any Aβ peptide, for example, Aβ₁₋₄₂ or Aβ₁₋₄₀, or combinations thereof.

The Aβ preparation with which the test immunological reagent is contacted can be derived from a variety of sources, for example, tissues, cell lines, synthesis, etc., that can provide an Aβ preparation with both Aβ monomers and one or more Aβ oligomers, that is substantially free of fibrils. In various embodiments, the Aβ preparation comprises a synthetically prepared preparation substantially free of fibrils which is treated with a crosslinking reagent.

A variety of crosslinking reagents can be used including, but not limited to, homofunctional group linkers and heterofunctional group linkers. The crosslinking reagent can be cleavable. Examples of homofunctional group linkers include, but are not limited to amine-amine linkers (e.g., glutaraldehyde; sebacic acid bis(N-succinimidyl) ester (DSS); and imidoester crosslinkers, such as, dimethyl adipimidate (DMA), dimethyl pimelimidate (DMP), dimethyl suberimidate (DMS), and dimethyl 3,3′-dithiobispropionimidate (DTBP)), thiol-thiol linkers (e.g., 1,4-bis[3-(2-pyridyldithio) propionamido]butane (DPDPB); bis[2-(N-succinimidyl-oxycarbonyloxy)ethyl] sulfone (BSOCOES); and ethylene glycol disuccinate di(N-succinimidyl) ester (EGS)), alcohol-alcohol linkers, carboxylic acid-carboxylic acid linkers, and aryl-aryl linkers (such as, for example, peroxynitrite; BDB; and metal-catalyzed oxidations). Examples of heterofunctional group linkers include, but are not limited to, amine-thiol linkers (e.g., m-maleimidobenzoyl-N-hydrosuccinamide (MBS)), amine-carboxylic acid linkers, amine-carbonyl linkers, thiol-alcohol linkers, thiol-carbonyl linkers, and thiol-carboxylic acid linkers.

The crosslinking reagent can be photoactive. Examples of photoactive crosslinking reagents include, but are not limited to, tris(2,2-bipyridyl)ruthenium(II) (which, upon illumination with light at a wavelength of 450 nm in the presence of an electron acceptor, induces dityrosine crosslinking); azidobenzoic acid (3-sulfo-N-succinimidyl) ester sodium salt (Sulfo-HSAB) (a photo-reactive amine-amine linker); and bis[2-(4-azidosalicylamido)ethyl] disulfide (BASED) (a photo-reactive amine-amine linker).

Crosslinking reagents can be chosen based on the amino acids linked. For example, in various embodiments, a tyrosine-tyrosine crosslinking reagent is used. Examples of tyrosine-tyrosine crosslinking reagents include, but are not limited to, peroxynitrite; BDB; metal-catalyzed oxidations; and tris(2,2-bipyridyl)ruthenium(II).

A. Aβ Preparations from Synthetic Aβ Peptide Sources

Suitable Aβ preparations for identifying immunological reagents having therapeutic efficacy can be prepared using synthetic Aβ peptides. In various embodiments, synthetically prepared soluble Aβ₁₋₄₂ can be used to prepare an Aβ preparation. In general, the synthetic soluble Aβ₁₋₄₂ approaches described herein, provide a more oligomeric Aβ preparation than the cell line approach. There are numerous publications on the preparation of synthetic Aβ oligomers, (see, e.g., Dahlgren et al., J. Biol. Chem., 277, p pp 32046-32053 (2002) and citations therein). These preparations are considered fibril- and protofibril-free by atomic force microscopy (AFM).

In general, a preparation of synthetic Aβ oligomers can be prepared by dissolving lyophilized Aβ₁₋₄₂ peptides in a solvent to de-aggregate the peptides and produce a solution of substantially unaggregated Aβ peptides. The Aβ peptides are then recovered from the solution and incubated in a culture media to produce a preparation of synthetic Aβ oligomers. In various embodiments, the synthetic Aβ oligomer preparation is used as the Aβ preparation in the methods and kits of the present invention. In some embodiments, the synthetic Aβ oligomer preparation is reacted with a crosslinking reagent, which modifies the oligomer profile of the Aβ oligomer preparation, to produce the Aβ preparation for use in the methods of the present invention.

Examples of solvents useful for de-aggregating the Aβ peptides include, but are not limited to, fluorinated alcohols. Fluorinated alcohols like hexafluoroisopropanol (HFIP) have been shown to break down β-sheet structure, disrupt hydrophobic forces in aggregated amyloid preparations, and promote α-helical secondary structure. HFIP is highly polar, miscible with water and many organic solvents, thermally stable, and transparent to UV light. HFIP exhibits strong hydrogen bonding and will associate with and dissolve most molecules with receptive sites such as oxygen, double bonds, or amine groups. For example, circular dichroism (CD) spectra of Aβ₁₋₄₂ and Aβ₁₋₄₀ solutions (prepared by dissolving lyophilized AP₁₋₄₂ peptide in HFIP) indicate a secondary structure which is almost entirely: 50-70% α-helical; 30-50% random coil, and <1% β-sheet. These results are also in agreement with the AFM analysis of Aβ1-42 solutions in HFIP that demonstrate the peptide assumes a uniform, unaggregated confirmation that shows no signs of aggregation after incubation for 24 hours.

Other solvents for use in the invention include any solvent capable of freeing the source Aβ of structural history (e.g., secondary, tertiary, etc. structure) obtained during the preparation and/or storage of the Aβ peptide.

B. Aβ Preparations from Cell Lines

An Aβ preparation can be derived from cells lines. In general, an Aβ preparation can be derived from a cell line expressing APP. The cell line can be cultured and Aβ peptides extracted from the cultured cells using techniques known to those of ordinary skill in the art. For example, APP-expressing cells such as Chinese hamster ovary (CHO) cells stably transfected with APP_{717V→F} (referred to as 7PA2 cells) can be cultured in Dulbecco's modified Eagle's medium (DMEM) with 10% fetal bovine serum, as described, for example, in Walsh et al., supra.

C. Aβ Preparations from Tissue Sources

An Aβ preparation can be derived from tissue sources. In general, brain tissues from an animal having an amyloidogenic disorder, engineered to express APP, or both, can be used as a source of Aβ peptides for use in producing an Aβ preparation. The tissues can be processed and Aβ peptides extracted from the processed tissue using techniques known to those of ordinary skill in the art. For example, tissues may homogenized in a denaturing buffer (e.g. a guanidine buffer comprising 5.0 M guanidine HCl/50 mM TrisCl, pH 8.0), diluted with casein buffer (e.g. 0.25% casein/0.05% sodium azide/20 μg/ml aprotinin/5 mM EDTA, pH 8.0/10 μg/ml leupeptin in PBS), and the homogenate centrifuged (e.g. 16,000×g for 20 min at 4° C.) to extract Aβ peptides (see, e.g., Johnson-Wood et al., Proc. Natl. Acad. Sci. USA 94:1550 (1997)). Non-denaturing extraction techniques that are widely known to those of skill in the art may also be employed in the preparation of soluble Aβ peptides from tissue sources (see, for example, Kuo, et al., J. Biol. Chem., 271: 4077 (1996); Lambert, et al., J. Neurochem. 79: 595 (2001)).

II. Identification of Immunoreagents Having Therapeutic Efficacy

The identification of an immunological reagent as having (or not having) therapeutic efficacy for the treatment of one or more amyloidogenic disorders, an ability to neutralize one or more neuroactive forms of Aβ, or both, or the prediction of an immunological reagent's therapeutic efficacy for the treatment of one or more amyloidogenic disorders, ability to neutralize one or more neuroactive forms of Aβ, or both is based, at least in part, on a comparison of the binding of one or more Aβ oligomers in the Aβ preparation to the immunological reagent as compared to the binding of Aβ monomers in the Aβ preparation to the immunological reagent. This comparison, for example, can be used to determine a relative binding of one or more Aβ oligomers as compared to Aβ monomers for the immunological reagent. In various embodiments, this relative binding is compared to the corresponding relative binding of one or more Aβ oligomers and Aβ monomers in the Aβ preparation to a control reagent.

In various embodiments, an immunological reagent is identified as having therapeutic efficacy for the treatment of one or more amyloidogenic disorders, an ability to neutralize one or more neuroactive forms of Aβ, or both, or predicted to have therapeutic efficacy for the treatment of one or more amyloidogenic disorders, ability to neutralize one or more neuroactive forms of Aβ, or both, when the binding of one or more Aβ oligomers in the Aβ preparation to the immunological reagent is greater than the binding of Aβ monomers in the Aβ preparation to the immunological reagent. The one or more Aβ oligomer for which binding is compared can include, for example, one or more of Aβ dimers, Aβ trimers, Aβ tetramers, and Aβ pentamers.

In one embodiment, an immunological reagent is identified as having therapeutic efficacy for the treatment of one or more amyloidogenic disorders, an ability to neutralize one or more neuroactive forms of Aβ, or both, when the binding of Aβ dimers in the Aβ preparation to the immunological reagent is greater than the binding of Aβ monomers in the Aβ preparation to the immunological reagent. In another embodiment, an immunological reagent is identified as having therapeutic efficacy for the treatment of one or more amyloidogenic disorders, an ability to neutralize one or more neuroactive forms of Aβ, or both, when the binding of Aβ trimers in the Aβpreparation to the immunological reagent is greater than the binding of Aβ monomers in the Aβ preparation to the immunological reagent. In yet another embodiment, an immunological reagent is identified as having therapeutic efficacy for the treatment of one or more amyloidogenic disorders, an ability to neutralize one or more neuroactive forms of Aβ, or both, when the binding of both Aβ dimers and Aβ trimers in the Aβ preparation to the immunological reagent is greater than the binding of Aβ monomers in the Aβ preparation to the immunological reagent.

In various embodiments, an immunological reagent is identified as having therapeutic efficacy for the treatment of one or more amyloidogenic disorders, an ability to neutralize one or more neuroactive forms of Aβ, or both when the ratio of the amount of Aβ monomer in the Aβ preparation which binds to the immunological reagent to the amount of one or more Aβ oligomers in the Aβ preparation which binds to the immunological reagent is less than one.

In various embodiments, an immunological reagent is identified as having therapeutic efficacy for the treatment of one or more arnyloidogenic disorders, an ability to neutralize one or more neuroactive forms of Aβ, or both, when the increased binding of one or more Aβ oligomers to the test immunological reagent as compared to Aβ monomers is an increased binding as compared to that for a control reagent contacted with the Aβ preparation. Such a control reagent can be an Aβ antibody known to have therapeutic efficacy for the treatment of one or more arnyloidogenic disorders, an ability to neutralize one or more neuroactive forms of Aβ, or both. For example, the amount of one or more Aβ oligomers in the Aβ preparation which bind to such a control reagent can be substantially equal to or greater than the amount of Aβ monomers in the Aβ preparation which bind to the control reagent.

In various embodiments, an immunological reagent is identified as having therapeutic efficacy for the treatment of one or more amyloidogenic disorders, an ability to neutralize one or more neuroactive forms of Aβ, or both, when the ratio of the amount of Aβ monomer in the Aβ preparation which binds to the immunological reagent to the amount of one or more Aβ oligomers in the Aβ preparation which bind to the immunological reagent is lower (e.g., lower than the typical margin of experimental error in repeat measurements of the same sample, expressed as one standard deviation from the mean of such measurements) than the corresponding binding amount ratio for a control reagent.

In various embodiments, an immunological reagent is identified as not having therapeutic efficacy, an ability to neutralize one or more neuroactive forms of Aβ, or both, when the binding of one or more Aβ oligomers in the Aβ preparation to the immunological reagent as compared to the binding of Aβ monomers in the Aβ preparation to the immunological reagent is less than the corresponding binding to a control reagent. Such a control reagent, for example, can be an Aβ antibody known to not have therapeutic efficacy for the treatment of the amyloidogenic disorder or disorders of interest. For example, the amount of one or more Aβ oligomers in the Aβ preparation which bind to such a control reagent can be substantially equal to or less than the amount of Aβ monomers in the Aβ preparation which bind to the control reagent.

In various embodiments, an immunological reagent is identified as having therapeutic efficacy for the treatment of one or more amyloidogenic disorders, an ability to neutralize one or more neuroactive forms of Aβ, or both, when the ratio of the amount of Aβ monomer in the Aβ preparation which binds to the immunological reagent to the amount of one or more Aβ oligomers in the Aβ preparation which bind to the immunological reagent is greater (e.g., greater than the typical margin of experimental error in repeat measurements of the same sample, expressed as one standard deviation from the mean of such measurements) than the corresponding binding amount ratio for a control reagent.

The binding of an Aβ species (i.e., Aβ monomers and Aβ oligomers) in the Aβ preparation to an immunological reagent or control reagent can be determined by a comparison of the amount of the given Aβ species in the Aβ preparation which binds to the reagent. The determination can be a qualitative, quantitative, or combination of both.

For example, the amount of Aβ monomers in an Aβ preparation which bind to a test immunological reagent and the amount of one or more Aβ oligomers in an Aβ preparation which bind to a test immunological reagent can be assessed using immunoprecipitation to precipitate from the Aβ preparation the Aβ monomers bound to the test immunological reagent and one or more Aβ oligomers bound to the test immunological reagent. The amount of Aβ monomer precipitate and the amount of precipitate for one or more Aβ oligomer species can be used to determine a relative binding, a relative bound amount, or both.

A wide variety of means can be used to determine the amount of one or more Aβ oligomers in the Aβ preparation which bind to a reagent (e.g., immunological reagent or control reagent) as compared to the amount of Aβ monomers in the Aβ preparation which bind to the reagent. In general, any technique capable of distinguishing Aβ monomers in the Aβ preparation which bind to the reagent from one or more Aβ oligomers in the Aβ preparation which bind to the reagent can be used. For example, one or more of immunoprecipitation, electrophoretic separation, and chromatographic separation (e.g. liquid chromatography), can be used to separate one or more Aβ species in the Aβ preparation which bind to the reagent.

The Aβ monomers in the Aβ preparation which bind to the reagent can be distinguished from one or more Aβ oligomers in the Aβ preparation which bind to the reagent by, for example, one or more of electrophoretic separation (e.g., on the immunoprecipitates, chromatographic fractions, etc.), chromatographic separation (e.g., on the immunoprecipitates, chromatographic fractions, excised electrophoresis bands, etc.), and mass spectrometry (e.g., on the immunoprecipitates, chromatographic fractions, excised electrophoresis bands, etc.).

In various embodiments, the amount of Aβ monomers and one or more Aβ oligomer species in an Aβ preparation which bind to a test immunological reagent is assessed using immunoprecipitation to precipitate from the Aβ preparation the Aβ monomers and one or more Aβ oligomer species bound to the test immunological reagent. In various embodiments, the immunoprecipitate is then subject to an electrophoretic separation (e.g., SDS-PAGE) to distinguish Aβ monomers from one or more Aβ oligomer species in the precipitate. The amount of Aβ monomers and Aβ oligomers present in the electrophoretic bands can be visualized, for example, using immunoblotting of the electrophoretic bands. The amount of precipitate for an Aβ species can be determined, for example, from the intensity of the corresponding electrophoretic bands, immunoblot bands, or a combination of both. The intensity determination can be qualitative, quantitative, or a combination of both.

Assessment of band intensity can be performed, for example, using appropriate film exposures which can be scanned and the density of bands determined with software, for example, AlphaEase software (AlphaInnotech) or ImageQuant software (Molecular Devices). Assessment of band intensity can be performed, for example, using any of a number of labels incorporated into the immunological reagent, an imaging reagent (e.g., an antibody used in an immunoblot), or both. Suitable labels include, but are not limited to, fluorescent labels, radioactive labels, paramagnetic labels, or combinations thereof.

In various embodiments, the amount of Aβ monomers and one or more Aβ oligomer species in an Aβ preparation which bind to a test immunological reagent can be assessed using mass spectrometry, for example, on the Aβ preparation itself a suitable time after it has been contacted with the test immunological reagent, or on Aβ monomers and one or more Aβ oligomer species bound to the test immunological reagent which have been extracted from the Aβ preparation.

In other aspects, the methods for identifying immunological reagents having therapeutic efficacy by contacting the Aβ preparation with an immunological reagent to be tested (test immunological reagent) and determining the affinity of the test immunological reagent for Aβ monomers in the Aβ preparation compared to the affinity of the test immunological reagent for affinity one or more Aβ oligomers in the Aβ preparation, such that an immunological reagent having therapeutic efficacy is identified based, at least in part, on the comparison. The one or more Aβ oligomers can include, for example, one or more of Aβ dimers, Aβ trimers, Aβ tetramers, or combinations thereof.

The affinity of the test immunological reagent (e.g., an Aβ antibody) for Aβ monomers in the Aβ preparation relative to its affinity for one or more of Aβ oligomers in the Aβ preparation, can also be compared, e.g., to that of a control reagent. For example, a suitable control reagent can exhibit a substantially equal affinity for Aβ monomers and one or more Aβ oligomers in the Aβ preparation. In various embodiments, an immunological reagent is identified as having therapeutic efficacy when the ratio of Aβ monomer affinity to Aβ oligomer affinity (or combination of oligomer affinities) for the immunological reagent is lower (e.g., lower than the typical margin of experimental error in repeat measurements of the same sample, expressed as one standard deviation from the mean of such measurements) than the corresponding affinity ratio for a control reagent.

In various embodiments, the relative ratio of Aβ monomer affinity to one of Aβ dimer, trimer or tetramer affinity is used, at least in part, to identify an immunological reagent as having therapeutic efficacy. In various embodiments, the relative ratio of Aβ monomer affinity to a combination of two or more of Aβ dimer, trimer and tetramer affinities is used, at least in part, to identify an immunological reagent as having therapeutic efficacy.

Labels can be used to assess the affinity of an immunological reagent for Aβ monomers, Aβ oligomers, or both. In various embodiments, a primary immunological reagent with affinity for Aβ is unlabelled and a secondary labeling agent is used to bind to the primary reagent. Suitable labels include, but are not limited to, fluorescent labels, paramagnetic labels, radioactive labels, and combinations thereof.

III. Contextual Fear Conditioning Assays

In various aspects, the present invention features methods of predicting, corroborating, or both, the results of animal assays for identifying immunological reagents having therapeutic efficacy for the treatment of one or more amyloidogenic disorders (e.g., Alzheimer's disease), or combination of amyloidogenic disorders. The assays are based, at least in part, on comparing cognition, as determined from a contextual fear conditioning study of the animal, before and after administration of a test immunological reagent to the animal.

Contextual fear conditioning is a common form of learning that is exceptionally reliable and rapidly acquired in most animals, for example, mammals. Test animals learn to fear a previously neutral stimulus because of its association with an aversive experience and/or environmental cue(s). (see, e.g., Fanselow, Anim. Learn. Behav. 18:264-270 (1990); Wehner et al., Nature Genet. 17:331-334. (1997); Caldarone et al., Nature Genet. 17:335-337 (1997)).

Contextual fear conditioning is especially useful for determining cognitive function or dysfunction, e.g., as a result of disease or a disorder, such as a neurodegenerative disease, e.g., Alzheimer's disease (AD), the presence of an unfavorable genetic alteration affective cognitive function (e.g., genetic mutation, gene disruption, or undesired genotype), and/or the efficacy of an agent, e.g., a recombinant antibody agent, on cognitive ability. Accordingly, the CFC assay provides a method for independently testing and/or validating the therapeutic effect of agents for preventing or treating cognitive disease, and in particular, a disease or disorder affecting one or more regions of the brains, e.g., the hippocampus, subiculum, cingulated cortex, prefrontal cortex, perirhinal cortex, sensory cortex, and medial temporal lobe.

Typically, the CFC assay is performed using standard animal chambers and the employment of conditioning training comprising a mild shock (e.g., 0.35 mA foot shock) paired with an auditory (e.g., a period of 85 db white noise), olfactory (e.g., almond or lemon extract), touch (e.g., floor cage texture), and/or visual cue (light flash). The response to the aversive experience (shock) is typically one of freezing (absence of movement except for respiration) but may also include eye blink, or change in the nictitating membrane reflex, depending on the test animal selected. The aversive response is usually characterized on the first day of testing to determine a baseline for unconditioned fear with aversive response results on subsequent test days, e.g., freezing in presence of the cue but in the absence of the aversive experience, being characterized as contextually conditioned fear. For improved reliability, test animals are typically tested separately by independent technicians and scored over time. Additional experimental design details can be found in the art, for example, in Crawley, J N, What's Wrong with my Mouse; Behavioral Phenotyping of Transgenic and Knockout Mice, Wiley-Liss, NY (2000).

Exemplary test animals (e.g., model animals) include mammals (e.g. rodents or non-human primates) that exhibit prominent symptoms or pathology that is characteristic of an amyloidogenic disorder such as Alzheimer's. Model animals may be created by selective inbreeding for a desired or they may genetically engineered using transgenic techniques that are well-known in the art, such that a targeted genetic alteration (e.g a genetic mutation, gene disruption) in a gene that is associated with the dementia disorder, leading to aberrant expression or function of the targeted gene. For example, several transgenic mouse strains are available that overexpress APP and develop amyloid plaque pathology and/or develop cognitive deficits that are characteristic of Alzheimer's disease (see for example, Games et al., supra, Johnson-Wood et al., Proc. Natl. Acad. Sci. USA 94:1550 (1997); Masliah E and Rockenstein E. (2000) J Neural Transm Suppl.;59:175-83).

Alternatively, the model animal can be created using chemical compounds (e.g. neurotoxins, anesthetics) or surgical techniques (e.g. stereotactic ablation, axotomization, transection, aspiration) that ablate or otherwise interfere with the normal function of an anatomical brain region (e.g. hippocampus, amygdala, perirhinal cortex, medial septal nucleus, locus coeruleus, mammalary bodies) or specific neurons (e.g. serotonergic, cholinergic, or dopaminergic neurons) that are associated with characteristic symptoms or pathology of the amyloidogenic disorder. In certain preferred embodiments, the animal model exhibits a prominent cognitive deficit associated with learning or memory in addition to the neurodegenerative pathology that associated with an amyloidogenic disorder. More preferably, the cognitive deficit progressively worsens with increasing age, such that the disease progression in the model animal parallels the disease progression in a subject suffering from the amyloidogenic disorder.

Contextual fear conditioning and other in vivo assays to test the functionality of the antibodies described herein may be performed using wild-type mice or mice having a certain genetic alteration leading to impaired memory or mouse models of neurodegenerative disease, e.g., Alzheimer's disease, including mouse models which display elevated levels of soluble Aβ in the brain, cerebrospinal fluid (CSF) or plasma. For example, animal models for Alzheimer's disease include transgenic mice that overexpress the “Swedish” mutation of human amyloid precursor protein (hAPPswe; Tg2576) which show age-dependent memory deficits and plaques (Hsiao et al. (1996) Science 274:99-102). The in vivo functionality of the antibodies described herein can also be tested using PDAPP transgenic mice, which express a mutant form of human APP (APP^{V71F}) and develop Alzheimer's disease at a young age (Bard, et al. (2000) Nature Medicine 6:916-919; Masliah E, et al. (1996) J Neurosci. 15; 16(18):5795-81 1). Other mouse models for Alzheimer's disease include the PSAPP mouse, a doubly transgenic mouse (PSAPP) overexpressing mutant APP and PS1 transgenes, described in Holcomb, et al. (1998) Nature Medicine 4:97-110, and the PS-1 mutant mouse, described in Duff, et al. (1996) Nature 383, 710-713. Other genetically altered transgenic models of Alzheimer's disease are described in Masliah E and Rockenstein E. (2000) J Neural Transm Suppl. 59:175-83.

IV Kits

The invention further provides kits for performing one or more methods or assays described above. Typically, such kits contain one or more of: reagents for preparing a suitable Aβ preparation or a suitable Aβ preparation. The kit can also contain one or more of a control reagent and a label. For quantification of the amount of an Aβ monomer or oligomer in an Aβ preparation which binds to, or is bound to, an immunological reagent; the label is typically in the form of labeled Aβ antibody. Kits also typically contain instructions providing directions for use of the kit. The instructions may also include a chart or other correspondence regime correlating levels of measured label with levels of antibodies to Aβ. The term instructions refer to any written or recorded material that is attached to, or otherwise accompanies a kit at any time during its manufacture, transport, sale or use. For example, the term instructions encompasses advertising leaflets and brochures, packaging materials, instructions, audio or videocassettes, computer discs, as well as writing imprinted directly on kits.

The present invention will be more fully described by the following non-limiting examples.

### EXAMPLES

### General Materials and Methods:

Preparation of Polyclonal and Monoclonal Aβ Antibodies

Anti-Aβ polyclonal antibodies were prepared from blood collected from two groups of animals. The first group consisted of 100 female Swiss Webster mice, 6 to 8 weeks of age. They were immunized on days 0, 15, and 29 with 100 μg of synthetic intact Aβ42 combined with Complete and Incomplete Freund's Adjuvants (CFA/IFA). A fourth injection was given on day 36 with one-half the dose of Aβ. Animals were exsanguinated upon sacrifice at day 42, serum was prepared and the sera were pooled to create a total of 64 ml. The second group consisted of 24 female mice, 6 to 9 weeks of age, isogenic with the PDAPP mice but nontransgenic for the human APP gene. They were immunized on days 0, 14, 28 and 56 with 100 μg of Aβ42 combined with CFA/IFA. These animals were also exsanguinated upon sacrifice at day 63, serum was prepared and pooled for a total of 14 ml. The two lots of sera were pooled. The antibody fraction was purified using two sequential rounds of precipitation with 50% saturated ammonium sulfate. The final precipitate was dialyzed against PBS and tested for endotoxin. The level of endotoxin was less than 1 EU/mg.

The anti-Aβ monoclonal antibodies were prepared from ascites fluid. The fluid was first delipidated by the addition of concentrated sodium dextran sulfate to ice-cold ascites fluid by stirring on ice to a final concentration of 0.238%. Concentrated CaCl₂ was then added with stirring to a final concentration of 64 mM. This solution was centrifuged at 10,000×g and the pellet was discarded. The supernatant was stirred on ice with an equal volume of saturated ammonium sulfate added dropwise. The solution was centrifuged again at 10,000×g and the supernatant was discarded. The pellet was resuspended and dialyzed against 20 mM Tris-HCl , 0.4 M NaCl, pH 7.5. This fraction was applied to a Pharmacia FPLC™ Sepharose Q™ Column and eluted with a reverse gradient from 0.4 M to 0.275 M NaCl in 20 mM Tris-HCl, pH 7.5.

The antibody peak was identified by absorbance at 280 nm and appropriate fractions were pooled. The purified antibody preparation was characterized by measuring the protein concentration using the bicinchoninic acid (BCA) assay by Pierce, a copper-based colorimetric protein assay (See Stoscheck, C M. (1990) Methods in Enzymology 182: 50-69). The purity was assessed using SDS-PAGE. The pool was also tested for endotoxin. The level of endotoxin was less than 1 EU/mg. Antibody titers less than 100 were arbitrarily assigned a titer value of 25.

### Example I

### Production and Characterization of Aβ Oligomers from Cell Line Sources

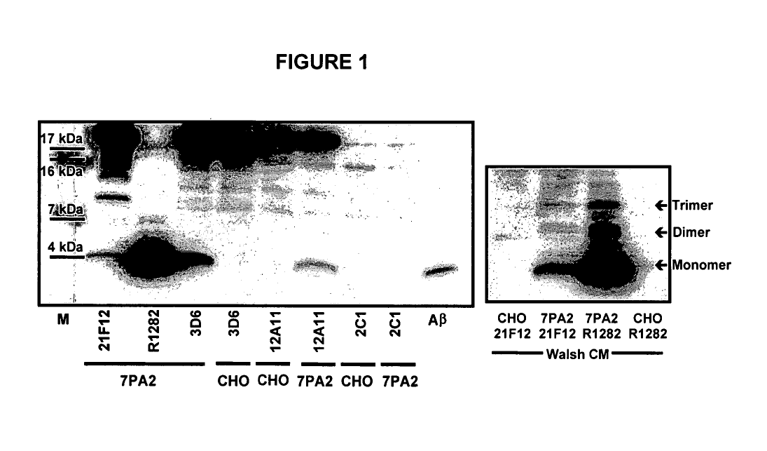

Aβ oligomeric species were profiled in conditioned media (CM) from cultured 7PA2 cells versus parental CHO cells. 7PA2 cells are CHO cells stably transfected with APP_{717V→F}. To profile the Aβ oligomers, CM was prepared from cells cultured in Dulbecco's modified Eagle's medium (DMEM) with 10% fetal bovine serum (FBS). The 7PA2 CM was immunoprecipitated with Aβ antibodies and imaged via Western blotting with 6E10 (Aβ epitope 6-10). FIG. 1 depicts profiles of the Aβ species in CM from 7PA2 cells as compared to CHO cell CM. The right panel depicts CM immunoprecipitated with the mAb 21F12 (Aβ-42) or the polyclonal Ab R1282. The left panel depicts CM immunoprecipitated with the mAbs 21F12, 3D6 (Aβ1-5), 12A11 (Aβ3-7) or 2C1 (Aβ 3-7), or the pAb R1282. In the right-hand panel (from left to right) lane 1 is a CHO CM control immunoprecipitated with Aβ antibody 21F12; lanes 2 and 3 are 7PA2 CM immunoprecipitated with Aβ antibodies 21F12 and R 1282, respectively; and lane 4 is CHO CM control immunoprecipitated with Aβ antibody R1282. CM immunoprecipitated in the right-hand panel and R1282 polyclonal antisera were provided by D. M. Walsh and D. J. Selkoe, prepared according to Walsh et al., supra. The monomer, dimer and trimer bands observed are noted in the figure. Monomeric, dimeric and trimeric Aβ species were readily detectable in 7PA2 CM immunoprecipitated with either the 21F12 antibody or the R1282 antibody.

In the left-hand panel, various monoclonal Aβ antibodies were tested for the ability to precipitate oligomeric Aβ. From left-to right, lane 1 (M) contains markers; lanes 2-4 are 7PA2 CM immunoprecipitated with Aβ antibodies 21F12, R1282 and 3D6, respectively; lanes 5 and 6 are CHO CM control immunoprecipitated with Aβ antibodies 3D6 and 12A11, respectively; lane 7 is 7PA2 CM immunoprecipitated with Aβ antibody 12A11; lane 8 is CHO CM control immunoprecipitated with Aβ antibody 2C1; lane 9 is 7PA2 CM immunoprecipitated with Aβ antibody 2C1; and lane 10 is synthetic Aβ42 peptide used as a standard. Monomeric and higher-ordered oligomeric Aβ species were readily detectable in 7PA2 CM immunoprecipitated with, for example, the 21F12, 3D6 and 12A11 monoclonal antibodies, as indicated.

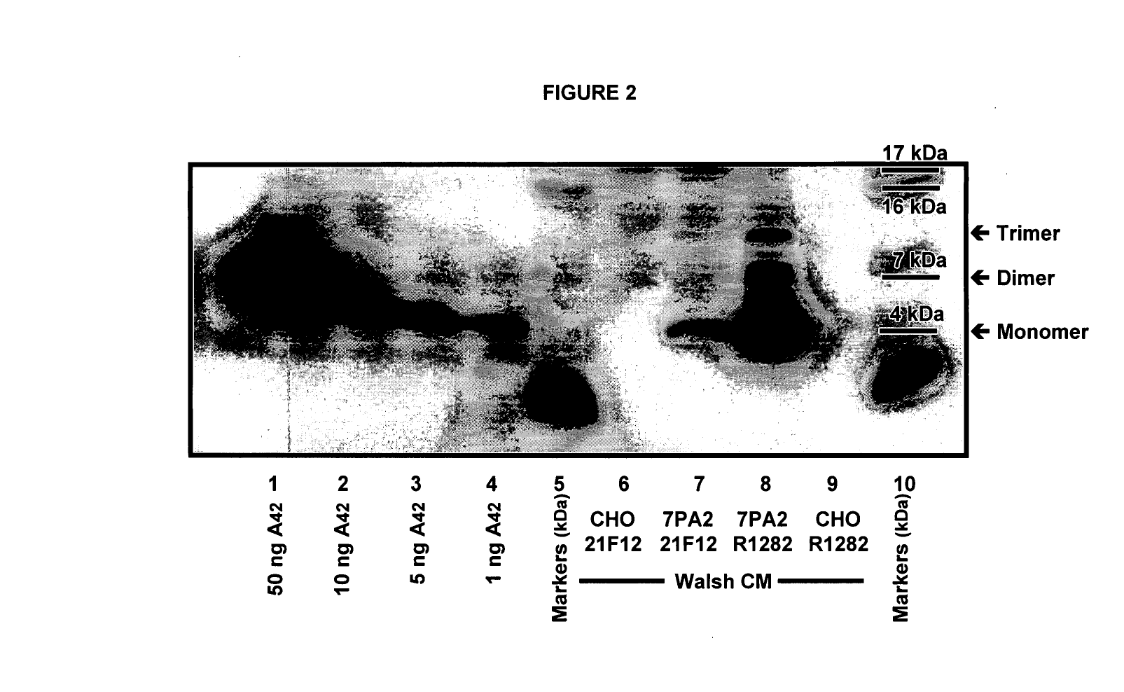

FIG. 2 compares the Aβ profile for 7PA2 and CHO CM immunoprecipitated with 21F12 or R1282 (lanes 6-9) with the profile for various amounts of synthetic Aβ₁₋₄₂: 50 nanograms (ng) in lane 1; 10 ng in lane 2; 5 ng in lane 3; and 1 ng in lane 4. Lanes 5 and 10 contain markers. The blot was imaged with 6E10. The data show that the Aβ monomer detected in CM migrates at the same position as Aβ monomer in the synthetic Aβ preparation and provides an approximation of the amount of monomeric Aβ present in the 7PA2 CM.

A further series of Aβ antibodies were tested for their ability to immunoprecipitate various Aβ species from 7PA2 CM, as described above. Table 1 provides a summary of the profiles generated using the various Aβ.

TABLE 1
Summary of 7PA2 CM Immunoprecipitations

| | ANTIBODY | Aβ | | |
|---|---|---|---|---|
| ANTIBODY | EPITOPE | Monomer | Dimer | Trimer |
| 3D6 | Aβ₁₋₅ | + | − | − |
| 2H3 | Aβ₂₋₇ | + | + | + |
| 6C6 | Aβ₂₋₁₀ | + | + | + |
| 10D5 | Aβ₃₋₆ | faint | − | − |
| | | band | | |
| 12B4 | Aβ₃₋₇ | + | + | + |
| 12A11 | Aβ₃₋₇ | + | − | − |
| 3A3 | Aβ₃₋₇ | faint | faint | faint |
| | | band | band | band |
| 2C1 | Aβ₃₋₇ | − | − | − |
| 266 | Aβ₁₆₋₂₃ | + | + | + |
| 22D12 | Aβ₁₈₋₂₂ | + | + | + |
| 6H9 | Aβ₁₉₋₂₃ | + | + | + |
| 2G3 | Aβ₃₃₋₄₀ | + | + | + |
| 16C11 | Aβ₃₃₋₄₂ | faint | − | − |
| | | band | | |
| 21F12 | Aβ₃₅₋₄₂ | + | + | + |
| R1282 | Aβ_{rabbit polyclonal} | + | + | + |

Several of the antibodies tested were able immunoprecipitate monomeric and higher-ordered oligomeric Aβ species from 7PA2 CM. Since the Aβ oligomer species concentration in the 7PA2 CM is relatively low, Aβ oligomers were generated using synthetic Aβ peptide as a starting source, described infra.

### Example II

### Aβ Preparations from Synthetic Aβ Peptide Sources

Aβ preparations (including monomeric Aβ and higher-ordered Aβ species) were prepared from synthetic Aβ peptide substantially as follows. Lyophilized Aβ₁₋₄₂ peptide was dissolved to 1 mM in 100% HFIP and separated into aliquots in microcentrifuge tubes. The HFIP frees the source Aβ peptide from any structural history (structure obtained during the preparation, purification and/or storage of the Aβ peptide). The HFIP was removed by evaporation, and lyophilization was used to remove residual HFIP to yield an Aβ peptide residue, typically in the form of a film. The Aβ peptide residue was stored (e.g., dessicated at −20° C.) for later use in preparing the Aβ preparation or used immediately.

For use, the Aβ peptide was resuspended in dimethyl sulfoxide (DMSO). The Aβ₁₋₄₂ in DMSO was added to Ham's F-12 (phenol red free) culture media to bring the peptide to a final concentration of 100 μM. The resultant solution was then incubated at 4° C. for 24 hours to produce the Aβ preparation. The preprations are considered fibril- and protofibril-free as determined by atomoc force microscopy (AFM).

### Example III

### Crosslinked Aβ Preparations from Synthetic Aβ Peptide Sources

An Aβ preparation was prepared from a synthetic Aβ source substantially as described in Example II followed by treatment of the resultant synthetic Aβ oligomers with peroxynitrite in the presence of sodium hydroxide (NaOH). Peroxynitrite crosslinks tyrosines and Aβ contains one tyrosine at position 10. Peroxynitrite was predicted to stabilize the preparation. For comparison purposes, a DMSO-solublized Aβ preparation was also evaluated. DMSO-solublized Aβ was prepared by dissolving Aβ, 42 peptide in DMSO followed by bath sonication for 30 minutes. The sonicated solution was immediately placed on dry ice and stored −80° C. until use.

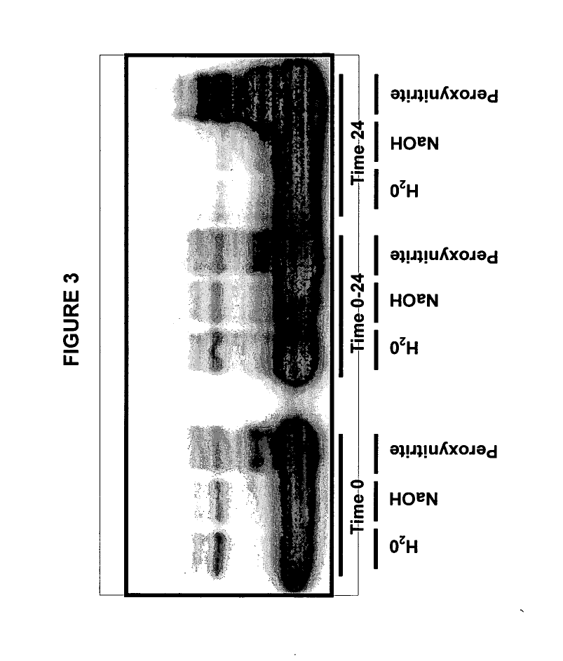

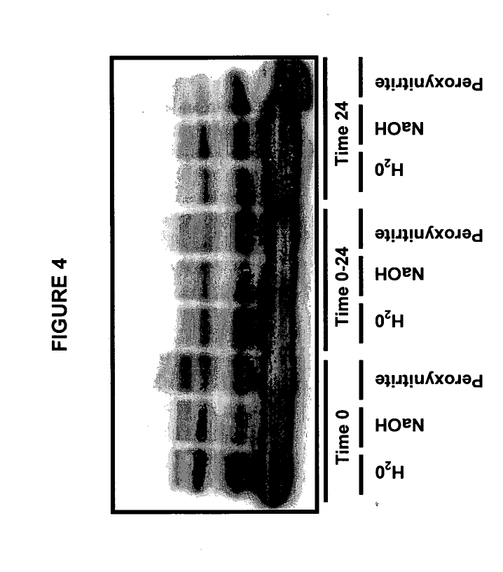

FIGS. 3 and 4 profile the peroxynitrited and DMSO-solubilized Aβ preparations, respectively. FIG. 3 depicts a Western blot of the peroxynitrited Aβ₁₋₄₂ preparation subject to various treatment conditions (water (H₂O), sodium hydroxide (NaOH), peroxynitrite) at various incubation times after treatment. FIG. 4 depicts a Western blot of the soluble Aβ₁₋₄₂ preparation subject to the various treatment conditions at various incubation times after treatment. Samples were diluted in sample buffer and separated by SDS-PAGE on a 12% NuPAGE gel. The protein was transferred to nitrocellulose membranes, the membranes boiled for 10 minutes in PBS (phosphate buffered saline) and then blocked overnight at 4° C. in a solution of TBS (tris buffered saline)/Tween/5% Carnation dry milk. Alternatively, membranes can be microwaved in PBS for 2 minutes, allowed to soak in the hot PBS for 3.5 minutes, and then microwaved again for 5 minutes. Membranes were then imaged with 6E10. For detection, membranes were incubated with anti-mouse Ig-HRP (horse radish peroxidase), developed using ECL Plus™ reagents (Amersham), and visualized using film. Molecular mass was estimated by SeeBlue Plus2™ (Invitrogen) molecular weight markers.

As the figures illustrate, the Aβ profile is similar for all treatment conditions with the soluble Aβ1-42 preparation. Monomeric and higher-ordered oligomeric Aβ species are all detectable. With the HFIP-solubilized Aβ preparation, the higher-ordered Aβ species were most abundant following peroxynitrite treatment for 24 hours. In particular, dimeric Aβ is readily detectable. Both preparations provide more abundant oligomeric sources for evaluating Aβ antibodies than CM from 7PA2 cells, described supra. The crosslinked preparation (from the HFIP-solubilized Aβ source) was chosen to further eveluate various Aβ antibodies for their ability to preferentially bind higher-ordered Aβ species (i.e., soluble, oligomeric Aβ).

### Example IV

### Identification of Aβ Antibodies Having Therapeutic Efficacy Using Aβ Preparations from Synthetic Aβ Peptide Sources

This example demonstrates the ability of various Aβ antibodies to preferentially bind to soluble, oligomeric Aβ. The data are used to predict the therapeutic efficacy of the Aβ antibodies.

In this Example, the Aβ preparation was prepared from synthetic Aβ substantially as follows:
(1) lyophilized Aβ₁₋₄₂ peptide was dissolved to 1 mM with ice cold 100% hexafluoroisopropanol (HFIP) (vortexed then incubated at room temperature for 1 hour) and separated into aliquots in microcentrifuge tubes (each tube containing 0.5 mg of Aβ₁₋₄₂ peptide);
(2) the HFIP was removed by evaporation followed by lyophilization to remove residual HFIP;
(3) the resultant Aβ peptide film/residue was stored, desiccated, at −20° C.;
(4) the Aβ peptide residue was resuspended in DMSO to a final concentration of 5 mM of peptide then added to ice cold Ham's F-12 (phenol red free) culture media to bring the peptide to a final concentration of 100 μM;
(5) the peptide was incubated at 4° C. for 24 h to produce synthetic Aβ oligomers at an approximately 100 μM concentration; and
(6) the synthetic Aβ oligomers were treated with peroxynitrite.

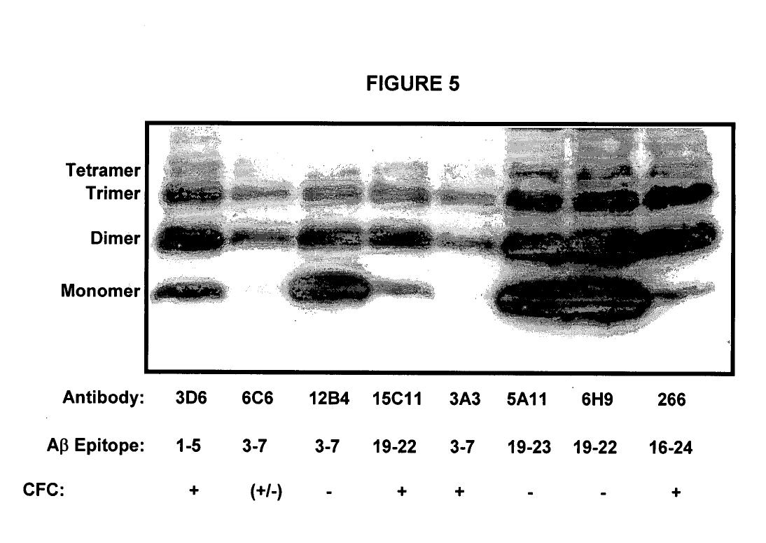

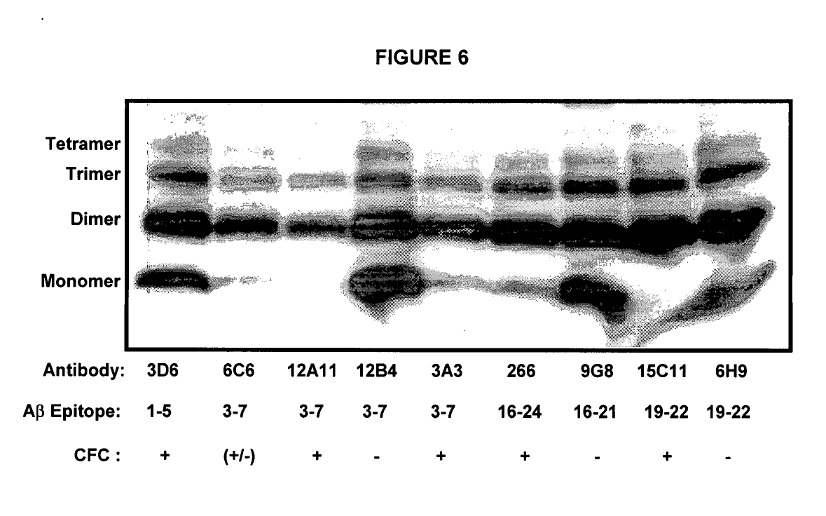

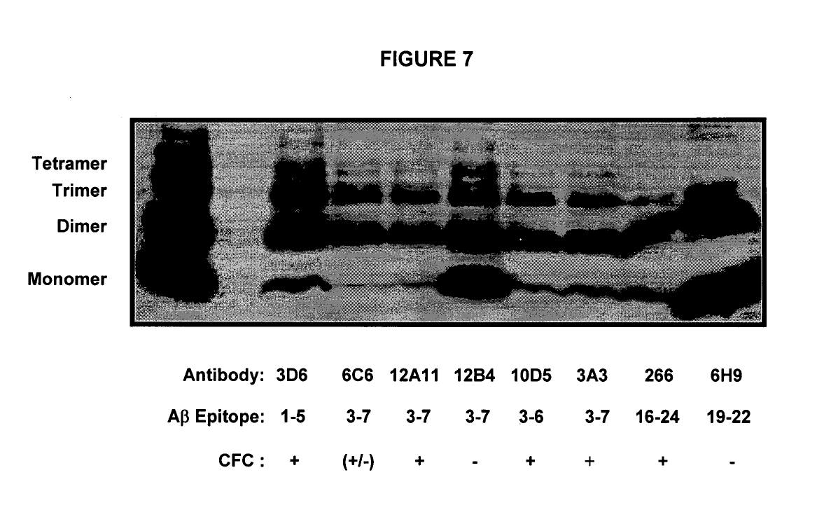

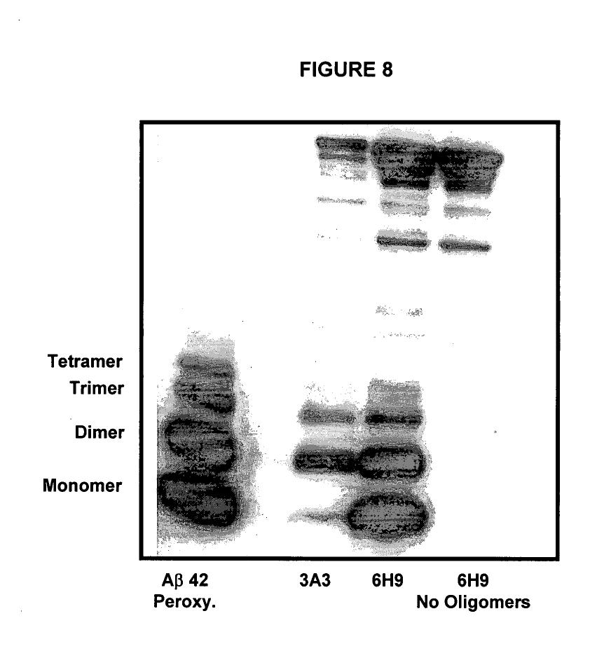

Aliquots of the Aβ preparation were then each contacted with a test immunological reagent, in this case antibodies, and the Aβ monomers and one or more Aβ oligomers which bound to the test immunological reagent were extracted from the Aβ preparation by immunoprecipitation. The various immunoprecipitates were separated by gel electrophoresis and immunoblotted (imaged) with the 3D6 antibody substantially as follows. Immunoprecipitate samples of FIGS. 5-8 were diluted in sample buffer and separated by SDS-PAGE on a 16% Tricine gel. The protein was transferred to nitrocellulose membranes, the membranes boiled in PBS, and then blocked overnight at 4° C. in a solution of TBS/Tween/5% Carnation dry milk. The membranes were then incubated with 3D6, a mouse monoclonal Aβ antibody to residues 1-5. For detection, the membranes were incubated with anti-mouse Ig-HRP, developed using ECL Plus™, and visualized using film. Molecular mass was estimated by SeeBlue Plus2™ molecular weight markers.

FIGS. 5-8 depict the results of contacting the above Aβ₁₋₄₂ preparations with various test immunological reagents (in FIGS. 5-8 Aβ antibodies) to determine the binding of, e.g., Aβ monomers, dimers, trimers, tetramers, pentamers, etc. in the Aβ preparation to the test immunological reagent. FIGS. 5-8 depict Western blots (imaged with 3D6) of immunoprecipitates of a peroxynitrite treated oligomeric Aβ preparation contacted with various Aβ antibodies. The approximate positions of Aβ₁₋₄₂ monomer, dimer, trimer and tetramer bands are indicated on the left-hand side of each figure. Indicated below each Aβ antibody is the Aβ epitope recognized by the antibody and CFC assay results (see Example V, supra) for the antibody, a “+” notation indicates an observation of increased cognition upon treatment with the antibody, a “−” notation indicates an observation of no change in cognition upon treatment with the antibody, a “+/−” notation indicates an observation of a trend of increased cognition upon treatment with the antibody but which is not statistically significant enough to be indicated as an observation of increased cognition.

Cell lines producing the antibodies 10D5 and 3D6, having the ATCC accession numbers PTA-5129 and PTA-5130, respectively, were deposited on Apr. 8, 2003, under the terms of the Budapest Treaty and cell lines producing the antibodies 6C6 and 9G8, having the ATCC accession numbers and ______ and ______, respectively, were deposited on Oct. 31, 2005, under the terms of the Budapest Treaty. Also, cell lines producing the antibodies 12A11, 5A11, 2H3, 15C11 and 3A3, having the ATCC accession numbers ______, ______, ______, ______, and ______, respectively, were deposited on , under the terms of the Budapest Treaty.

In FIGS. 5-8, an increased binding of an Aβ antibody for Aβ dimers or higher ordered oligomers in the Aβ preparation, relative to the binding of the Aβ antibody for Aβ monomers in the Aβ preparation, predicts that the Aβ antibody has therapeutic efficacy for the treatment of Alzheimer's disease. Notably, Aβ antibodies 3D6, 15C11, 10D5, 12A11 and 266 are predicted to have therapeutic efficacy for the treatment of Alzheimer's disease. A further antibody, 3A3 is also predicted to have therapeutic efficacy for the treatment of Alzheimer's disease.

### Example V

### Contextual Fear Conditioning Assay in Transgenic Mice

CFC assays were conducted as described as described in U.S. Ser. No. 60/636,842, filed Dec. 15, 2004, U.S. Ser. No. 60/637,253, filed Dec. 16, 2004, and U.S. Ser. No. 60/736,119, filed on Nov. 10, 2005. Briefly, Tg2576 mice (overexpressing the Swedish mutation of the amyloid precursor protein) were trained and tested on two consecutive days. Training consisted of administering an auditory cue with concurrent foot shock (pattern repeated twice consecutively) on day 1. Testing involved assaying for contextual memory on day 2. Administration of PBS or antibodies was performed prior to training in order that serum levels of antibody be significant during the memory consolidation phase. Antibodies were administered by intraperitoneal injection. Animals were scored for contextual memory deficit reversal (contextual memory in test animals versus PBS-treated animals). The results are summarized in Table 2.

TABLE 2

| | | | Memory Deficit | | Impairment | |
|---|---|---|---|---|---|---|
| | | | Reversal | | Status | |
| Treatment | Treatment | Antibody | 30 | 10 | 30 | 10 |
| Group | Antibody | Specificity | mg/kg | mg/kg | mg/kg | mg/kg |
| 1 | 3D6 | 1-5 | + | − | + | + |
| 2 | 6C6 | 3-7 | +/− | ND | − | ND |
| 3 | 10D5 | 3-6 | + | − | +/− | − |
| 4 | 12B4 | 3-7 | − | ND | +/− | ND |
| 5 | 12A11 | 3-7 | + | + | + | + |
| 6 | 266 | 16-24 | ND | + | ND | + |
| 7 | 6H9 | 19-22 | − | ND | − | ND |
| 8 | 15C11 | 19-22 | + | − | + | + |

Regarding memory deficit reversal, the “+” notation indicates significant memory deficit reversal upon treatment with the antibody, the “−” notation indicates an observation of no memory deficit reversal upon treatment with the antibody, and the “+/−” notation indicates an observation of a trend towards memory deficit reversal upon treatment with the antibody. The 12A11 antibody was also determined to cause significant memory deficit reversal at 1.0 mg/kg and 0.3 mg/kg dosages (data not shown).

Regarding impairment status, the “+” notation indicates no significant memory impairment and the “+/−” notation indicates a trend towards no impairment. Animals treated with the 10D5-antibody also exhibited no significant memory impairment at the 3 mg/kg dosage (data not shown). Animals treated with the 266 antibody also exhibited significant memory deficit reversal at a 3 mg/kg dosage.

The results of the CFC assay indicate that the 3D6, 3A3, 10D5, 12A11, 266 and 15C11 antibodies exhibit significant therapeutic efficacy with the 6C6 and 12B4 antibodies also exhibiting some efficacy. Notably, the 3D6, 3A3, 15C11, 266, 12A11 and 10D5 antibodies were also significant when tested in the assays of the invention (see e.g., Example IV, supra.)

### Example VI

### Aβ Preparations from Tissue Sources

An Aβ preparation is prepared from APP transgenic mouse brain tissue as follows. The APP transgenic mice brain tissue is homogenized in 10 volumes of ice-cold guanidine buffer (5.0 M guanidine-HCl, 50 mM Tris-HCl, pH 8.0). The homogenates are then mixed (e.g., by gentle agitation using an Adams Nutator™ (Fisher)) for three to four hours at room temperature to produce analytes. The analytes are stored, prior to extraction of Aβ peptides, at −20° C. The Aβ peptides are then extracted using techniques known to the art to produce an Aβ preparation. (see, e.g., Johnson-Wood et al., supra).

### Example VII

### Aβ Fibril Preparations from Synthetic Aβ Peptide Sources Synthetic Aβ fibrils are prepared substantially as follows. Lyophilized

Aβ₁₋₄₂ peptide is dissolved to 1 mM in 100% HFIP and separated into aliquots in microcentrifuge tubes. The HFIP is removed by evaporation, and lyophilization is used to remove residual HFIP, to yield an Aβ peptide residue, typically in the form of a film. The Aβ peptide residue is stored (e.g., dessicated, at −20° C.) for later use in preparing synthetic Aβ fibrils or used immediately. A synthetic Aβ fibril preparation is prepared from the Aβ peptide residue by resuspending the Aβ peptide residue in dry (CH₃)₂SO (DMSO) to 5 mM, diluting in 10 mM HCl to bring the Aβ peptide to a final concentration of 100 μM and incubating the peptide at 37° C. for 24 h to produce synthetic Aβ fibrils. Aβ fibrils so-prepared are used, for example, in studying the role of Aβ oligomers in fibril formation or in comparing an antibody's specificity for oligomeric Aβ species versus Aβ fibrillar species.

Although the foregoing invention has been described in detail for purposes of clarity of understanding, it will be obvious that certain modifications may be practiced within the scope of the appended claims. All publications and patent documents cited herein, as well as text appearing in the figures and sequence listing, are hereby incorporated by reference in their entirety for all purposes and to the same extent as if each were so individually denoted.

We claim:

1. A method for identifying an immunological reagent having therapeutic efficacy, comprising the steps of:
contacting an Aβ preparation with a test immunological reagent, the Aβ preparation comprising Aβ monomers and one or more Aβ oligomers; and
determining an increased binding of the test immunological reagent to the one or more Aβ oligomers as compared to the Aβ monomers, such that an immunological reagent having therapeutic efficacy is identified.

2. The method of claim 1, wherein the Aβ preparation has been treated with a crosslinking reagent.

3. The method of claim 2, wherein the crosslinking reagent is a tyrosine crosslinking reagent.

4. The method of claim 2, wherein the crosslinking reagent is peroxynitrite.

5. The method of claim 1, wherein the test immunological reagent is an Aβ antibody.

6. The method of claim 1, wherein the therapeutic efficacy is an efficacy in treating an amyloidogenic disorder.

7. The method of claim 6, wherein the amyloidogenic disorder is one or more of systemic amyloidosis, Alzheimer's disease, cerebral amyloid angiopathy, mature onset diabetes, Parkinson's disease, Huntington's disease, fronto-temporal dementia, and the prion-related transmissible spongiform encephalopathies (kuru and Creutzfeldt-Jacob disease in humans and scrapie and BSE in sheep and cattle, respectively), and combinations thereof.

8. The method of claim 7, wherein the amyloidogenic disorder is Alzheimer's disease.

9. The method of claim 1, wherein the Aβ oligomers comprise Aβ dimers.

10. The method of claim 1, wherein the Aβ oligomers comprise Aβ trimers.

11. The method of claim 1, wherein the Aβ oligomers comprise both Aβ dimers and Aβ trimers.

12. The method of claim 1, wherein the step of contacting the test immunological reagent with the Aβ preparation comprises immunoprecipitating the test immunological reagent.

13. The method of claim 12, wherein the step of determining an increased binding comprises performing an electrophoretic separation on the immunoprecipitated reagent.

14. The method of claim 13, wherein the step of determining an increased binding further comprises immunodetection of the immunoprecipitated reagent.

15. The method of claim 14, wherein the immunodetection is achieved using an antibody which detects Aβ monomers and Aβ oligomers.

16. The method of claim 15, wherein the antibody which detects Aβ monomers and Aβ oligomers is labeled using one or more of a fluorescent label, radioactive label, paramagnetic label, or combinations thereof.

17. The method of claim 1, wherein the immunological reagent is identified as having therapeutic efficacy when the increased binding of one or more Aβ oligomers to the test immunological reagent compared to the Aβ monomers is an increased binding as compared to that for a control reagent contacted with the Aβ preparation.

18. The method of claim 17, wherein the amount of Aβ monomers bound to the control reagent is substantially equal to the amount of one or more Aβ oligomers bound to the control reagent.

19. The method of claim 18, wherein the amount of Aβ monomers bound to the control reagent is greater than the amount of one or more Aβ oligomers bound to the control reagent.

20. The method of claim 18, wherein the amount of Aβ monomers bound to the control reagent is less than the amount of one or more Aβ oligomers bound to the control reagent.

21. A kit for performing the method of claim 1, the kit comprising one or more of:
reagents for preparing an Aβ preparation, and an Aβ antibody.

22. A method for identifying an immunological reagent having therapeutic efficacy for the treatment of an amyloidogenic disorder, comprising the steps of:
precipitating at least a portion of an Aβ preparation with an immunological reagent, the Aβ preparation comprising Aβ monomers and one or more Aβ oligomers;
comparing the amount of precipitated Aβ monomer to the amount of precipitated Aβ oligomers; and
identifying the immunological reagent as having therapeutic efficacy for the treatment of the amyloidogenic disorder based at least on the amount of Aβ monomer relative to the amount of Aβ oligomers.

23. The method of claim 22, wherein the Aβ preparation has been treated with a crosslinking reagent.

24. The method of claim 23, wherein the crosslinking reagent is a tyrosine crosslinking reagent.

25. The method of claim 23, wherein the crosslinking reagent is peroxynitrite.

26. The method of claim 22, wherein the immunological reagent is an Aβ antibody.

27. The method of claim 22, wherein the therapeutic efficacy is an efficacy in treating an amyloidogenic disorder.

28. The method of claim 27, wherein the amyloidogenic disorder is one or more of systemic amyloidosis, Alzheimer's disease, cerebral amyloid angiopathy, mature onset diabetes, Parkinson's disease, Huntington's disease, fronto-temporal dementia, and the prion-related transmissible spongiform encephalopathies (kuru and Creutzfeldt-Jacob disease in humans and scrapie and BSE in sheep and cattle, respectively), and combinations thereof.

29. The method of claim 28, wherein the amyloidogenic disorder is Alzheimer's disease.

30. The method of claim 22, wherein the Aβ oligomers comprise Aβ dimers.

31. The method of claim 22, wherein the Aβ oligomers comprise Aβ trimers.

32. The method of claim 22, wherein the Aβ oligomers comprise both Aβ dimers and Aβ trimers.

33. The method of claim 22, wherein the step of comparing comprises performing an electrophoretic separation of the precipitated Aβ monomer and Aβ oligomers.

34. The method of claim 33, wherein the step of comparing further comprises immunodetection of the immunoprecipitated reagent following the electrophoretic separation.

35. The method of claim 34, wherein the immunodetection is achieved using an antibody which detects Aβ monomers and Aβ oligomers.

36. The method of claim 35, wherein the antibody which detects Aβ monomers and Aβ oligomers is labeled using one or more of a fluorescent label, radioactive label, paramagnetic label, or combinations thereof.

37. The method of claim 22, wherein the immunological reagent is identified as having therapeutic efficacy when the amount of Aβ monomer is less than the amount of one or more Aβ oligomers.

38. The method of claim 37, wherein the immunological reagent is identified as having therapeutic efficacy when the amount of Aβ monomer is less than the amount of Aβ dimers.

39. The method of claim 37, wherein the immunological reagent is identified as having therapeutic efficacy when the amount of Aβ monomer is less than the amount of Aβ trimers.

40. The method of claim 22, wherein the immunological reagent is identified as having therapeutic efficacy based on a low amount of Aβ monomer relative to the amount of Aβ oligomers, as compared to corresponding relative amounts of Aβ monomers to Aβ oligomers precipitated by a control reagent contacted with the Aβ preparation.

41. The method of claim 40, wherein the amount of Aβ monomer is high relative to the amount of Aβ oligomers precipitated by the control reagent.

42. A kit for performing the method of claim 22, the kit comprising a synthetic Aβ preparation and one or more reagents for preparing an Aβ oligomer preparation and instructions for preparing the Aβ oligomer preparation.

43. The kit of claim 42, wherein one or more of the one or more reagents comprises a crosslinking reagent.

44. A method for identifying an immunological reagent having the ability to neutralize one or more neuroactive forms of Aβ, comprising the steps of:
contacting an Aβ preparation with a test immunological reagent, wherein the Aβ preparation comprises Aβ monomers and one or more Aβ oligomers; and
determining an increased binding of the test immunological reagent to the Aβ oligomers as compared to the Aβ monomers, such that an immunological reagent having the ability to neutralize one or more neuroactive forms of Aβ is identified.

45. The method of claim 44, wherein the neuroactive Aβ species comprise Aβ dimers, Aβ trimers, Aβ tetramers, Aβ pentamers, or combinations thereof.

46. The method of claim 44, wherein the neuroactive Aβ species comprise Aβ dimers.

47. The method of claim 44, wherein the Aβ preparation has been treated with a crosslinking reagent.

48. The method of claim 47, wherein the crosslinking reagent is a tyrosine crosslinking reagent.

49. The method of claim 47, wherein the crosslinking reagent is peroxynitrite.

50. The method of claim 44, wherein the test immunological reagent is an Aβ antibody.

51. The method of claim 44, wherein the step of contacting the test immunological reagent with the Aβ preparation comprises immunoprecipitating the test immunological reagent.

52. The method of claim 51, wherein the step of determining an increased binding comprises performing an electrophoretic separation on the immunoprecipitated reagent.

53. The method of claim 52, wherein the step of determining an increased binding further comprises immunodetection of the immunoprecipitated reagent.

54. The method of claim 53, wherein the immunodetection is achieved using an antibody which detects Aβ monomers and Aβ oligomers.

55. The method of claim 54, wherein the antibody which detects Aβ monomers and Aβ oligomers is labeled using one or more of a fluorescent label, radioactive label, paramagnetic label, or combinations thereof.

56. The method of claim 44, wherein the immunological reagent is identified as having the ability to neutralize one or more neuroactive forms of Aβ when the increased binding of one or more Aβ oligomers to the test immunological reagent compared to the Aβ monomers is an increased binding as compared to that for a control reagent contacted with the Aβ preparation.

57. The method of claim 56, wherein the control reagent immunoprecipitates an amount of Aβ monomer that is high relative to the amount of Aβ oligomers.

58. A method for identifying an immunological reagent having therapeutic efficacy for the treatment of an amyloidogenic disorder, comprising the steps of:
contacting an Aβ preparation with an immunological reagent, wherein the Aβ preparation comprises Aβ monomers and one or more Aβ oligomers;
comparing the amount of Aβ monomer bound to the immunological reagent to the amount of Aβ oligomers bound to the immunological reagent to determine a relative bound amount; and
identifying the immunological reagent as having therapeutic efficacy for the treatment of the amyloidogenic disorder based at least on the relative bound amount.

59. An in vitro assay for identifying the results of an animal assay for identifying immunological reagents having therapeutic efficacy for the treatment of one or more amyloidogenic disorders comprising the steps of:
contacting an Aβ preparation with an immunological reagent, wherein the Aβ preparation comprises Aβ monomers and one or more Aβ oligomers;
comparing the amount of Aβ monomer bound to the immunological reagent to the amount of Aβ oligomers bound to the immunological reagent to determine a relative bound amount; and
identifying that a test animal administered the immunological agent will evidence a post-administration level of cognition that is greater than a pre-administration level of cognition if the amount of Aβ monomer bound to the immunological reagent is less than the amount of Aβ oligomers bound to the immunological reagent.

60. A method for identifying an immunological reagent having the ability to effect a rapid improvement in cognition in an animal, comprising the steps of:
contacting an Aβ preparation with a test immunological reagent, wherein the Aβ preparation comprises Aβ monomers and one or more Aβ oligomers;
determining an increased binding of the test immunological reagent to the Aβ oligomers as compared to the Aβ monomers, such that an immunological reagent having the ability to effect a rapid improvement in cognition in an animal is identified; and
confirming in a test animal a rapid improvement in cognition.

61. The method of claim 60, wherein the animal is a human.

62. The method of claim 60, wherein the test animal is an animal model for Alzheimer's Disease tested in contextual fear conditioning (CFC).
